(19) United States
(12) Patent Application Publication
(10) Pub. No.: US 2024/0159718 A1
(43) Pub. Date: May 16, 2024

(54) SIZE EXCLUSION CHROMATOGRAPHY ANALYSIS OF EMPTY AND FULL AAV CAPSIDS

(71) Applicant: Genzyme Corporation, Cambridge, MA (US)

(72) Inventor: He MENG, Newton, MA (US)

(21) Appl. No.: 18/282,739

(22) PCT Filed: Mar. 21, 2022
(86) PCT No.: PCT/US2022/071245

§ 371 Date: Sep. 18, 2023

Related U.S. Application Data
(60) Provisional application No. 63/164,206, filed on Mar. 22, 2021.

(51) Int. Cl. G01N 30/74; C12N 15/86; G01N 30/86; G01N 30/88

(52) U.S. Cl. CPC G01N 30/74; C12N 15/86; G01N 30/8624; G01N 30/88; C12N 2750/14143; C12N 2750/14151; G01N 2030/027

(57) ABSTRACT

Provided herein are methods to determine the relative amount of empty and/or partial capsids in compositions comprising recombinant adeno-associated virus (rAAV) particles. The methods utilize chromatography (e.g., size exclusion chromatography) with dual wavelength detection.

### CROSS-REFERENCE TO RELATED APPLICATIONS

This application claims the priority benefit of U.S. Provisional Application No. 63/164,206, filed Mar. 22, 2021, which is incorporated herein by reference in its entirety.

### FIELD OF THE INVENTION

The present invention relates to methods to analyze relative levels of full and empty capsids in compositions of recombinant adeno-associated virus (rAAV) particles. The methods relate to size exclusion chromatography with dual wavelength detection.

### BACKGROUND OF THE INVENTION

Adeno-associated virus (AAV) is a small nonpathogenic parvovirus, in which a linear single stranded DNA genome with a size of approximately 4.7 kilobases is packaged into a nonenveloped icosahedral capsid. The viral genome consists of three viral genes rep (replication), cap (capsid), and aap (assembly-activating protein) flanked by two inverted terminal repeats (ITRs) (Naso, M. F. et al., BioDrugs 2017, 31 (4), 317-334). The capsid is assembled as a 60-mer from three viral proteins (VPs) VP1, VP2, and VP3 with an approximately 5:5:50 ratio (Daya, S. et al., Clin Microbiol Rev 2008, 21 (4), 583-93). In therapeutic recombinant AAVs (rAAVs), the viral genome is replaced with a transgene, while the ITRs are retained for proper genome replication and packaging. The transgene is converted into a double stranded DNA for gene expression after entering the target cell nucleus, to exert therapeutic effects.

To date, three rAAV gene therapies have been approved: Glybera® for rare lipoprotein lipase deficiency (EMA, 2012); Luxturna® for Leber's congenital amaurosis (FDA, 2017) and Zolgensma® for spinal muscular atrophy (FDA, 2019) (Li, C. and Samulski, R. J., Nat Rev Genet 2020, 21 (4), 255-272). Many more rAAVs are in clinical trials for various disease indications (Li, C. and Samulski, R. J., Nat Rev Genet 2020, 21 (4), 255-272; Russell, S. et al., Lancet 2017, 390 (10097), 849-860; Wang, D. et al., Nature reviews. Drug discovery 2019, 18 (5), 358-378). During manufacturing, vectors harboring full-length genome (full capsids), no genome (empty capsids), fragments of genome (partial capsids), and non-transgene related host cell DNAs are produced (Wright, J. F., Biomedicines 2014, 2 (1), 80-97). While bonafide full capsids are purified as desired product, empty capsids cannot be easily removed and are inevitably present in the drug substance (DS) due to their similar structural properties (Naso, M. F. et al., BioDrugs 2017, 31 (4), 317-334; Qu, G. et al., J Virol Methods 2007, 140 (1-2), 183-92; Wright, J. F., Gene Ther 2008, 15 (11), 840-8). Although the impact of the presence of empty capsids on therapeutic outcomes is not fully understood yet, Gao et al. reported that empty capsids reduced transduction efficiency and induced liver transaminitis in mouse models (Gao, K. et al., Mol Ther Methods Clin Dev 2014, 1 (9), 20139). In contrast, Mingozzi et al. suggested that empty capsids can serve as decoys to mitigate the inhibitory effect of pre-existing anti-AAV antibodies and actually enhance gene transfer efficiency (Mingozzi, F. et al., Science translational medicine 2013, 5 (194), 194ra92; Wright, J. F., Mol Ther 014, 22 (1), 1-2). Independent of the impact of empty capsids, the level of empty capsids needs to be monitored to further the field's understanding of their impact on safety and efficacy and controlled to ensure the consistency of product quality (Schnodt, M. et al., Hum Gene Ther Methods 2017, 28 (3), 101-108; Flotte, T. R., Hum Gene Ther 2017, 28 (2), 147-148; EMA, Guideline on the quality, non-cinical and clinical aspects of gene therapy medicinal products 2018; p EMA/CAT/80183/2014; FDA, Guidance for Industry Chemistry, Manufacturing, and Control (CMC) Information for Human Gene Therapy Investigational New Drug Applications (INDs) 2020).

Multiple methods have been reported for quantifying empty or full capsids in AAVs. Sommer et al. quantified empty capsids in purified AAV samples based on the A₂₆₀/A₂₈₀ ratio from optical density measurements after sample denaturation. The capsid-to-vector genome ratios (cp/vg) correlated well with the ratios determined by qPCR and capsid ELISA (Sommer, J. M. et al., Mol Ther 2003, 7 (1), 122-8). However, since the specificity of the method is limited, non-vector proteins, free nucleic acids, and other components can significantly affect the accuracy of this method. Transmission electron microscopy (TEM) has been used to visualize AAV particles after negative staining (Takahashi, E. et al., Analytical biochemistry 2006, 349 (2), 208-17). Empty and full capsids are differentiated based on the electron density and counted to yield percent full capsids. Burnham et al. employed analytical ultracentrifugation sedimentation velocity (AUC-SV) experiments to characterize AAV vectors based on their sedimentation behavior in a centrifugal field (Burnham, B. et al., Hum Gene Ther Methods 2015, 26 (6), 228-42). In addition to empty, full, and partial capsids, AUC-SV can also resolve higher order species and fragmented capsids. Pierson et al. used charge detection mass spectrometry (CDMS) to determine the distribution of empty, partial and full capsids by concurrently measuring the mass charge ratio (m/z) and the charge (z) of individual ions (Pierson, E. E. et al., Anal Chem 2016, 88 (13), 6718-25). Li et al. has recently introduced a capillary isoelectric focusing (cIEF) method to determine the empty and full ratio of AAV vectors (Li, T. et al., Curr Mol Med 2020). These methods distinguish the empty and full capsids using different mechanisms, and therefore provide complementary insights in the composition of the sample. However, some methods such as AUC and CDMS are difficult to implement in a quality control (QC) environment, and some may face challenges due to inadequate assay range or low throughput. Chromatographic methods, being high-throughput, as well as readily accessible and deployed in QC and development labs, can therefore be useful for determining the level of empty and/or full capsids.

Anion exchange chromatography (AEX) has been used for determining the level of empty capsids in several serotypes by exploring the minor difference in isoelectric point (pI) between empty and full capsids. Lock et al. resolved density gradient purified AAV8 empty capsids from full capsids using a fast flow liquid chromatography instrument equipped with a CIM-QA monolithic disk (Lock, M. et al., Hum Gene Ther Methods 2012, 23 (1), 56-64). Fu et al. separated empty capsids of a nondisclosed serotype and affinity purified AAV over a CIMac AAV full/empty-0.1 analytical column (Fu, X. et al. Hum Gene Ther Methods 2019, 30 (4), 144-152). Wang et al. optimized the separation of empty capsids of an AAV6.2 sample over the CIMac AAV full/empty-0.1 analytical column and employed an empirical response conversion factor to quantitate empty capsids based on the fluorescence signal (Wang, C. et al., Mol Ther Methods Clin Dev 2019, 15, 257-263). Although these successes highlight the utility of AEX methods for quantitation of empty capsids of serotype specific AAVs, they also clearly show that the separation conditions for each serotype need to be carefully optimized.

What is needed is an assay using readily accessible instruments for high throughput analysis of rAAV compositions.

All references cited herein, including patent applications and publications, are incorporated herein by reference in their entireties.

### BRIEF SUMMARY

In some aspects, the invention provides methods to determine the presence of empty and/or partial capsids in a composition comprising recombinant adeno-associated virus (rAAV) particles, the method comprising: a) subjecting the composition to chromatography under conditions to separate the rAAV particles from impurities in the composition in an eluate of the chromatography; b) measuring the UV absorbance at about 250 nm to about 270 nm (A₂₅₀₋₂₇₀) and about 220 nm to about 240 nM (A₂₂₀₋₂₄₀) of the eluate, c) plotting chromatograms of the A₂₅₀₋₂₇₀ and A₂₂₀₋₂₄₀ of the eluate, d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the presence of empty and/or partial capsids in the composition.

In some aspects, the invention provides methods of measuring the relative amount of empty and/or partial capsids in a composition comprising rAAV particles, the method comprising: a) subjecting the composition to chromatography under conditions to separate the rAAV particles from impurities in the composition in an eluate of the chromatography; b) measuring the UV absorbance at about 250 nm to about 270 nm (A₂₅₀₋₂₂₀) and about 220 nm to about 240 nM (A₂₂₀₋₂₄₀) of the eluate, c) plotting chromatograms of the A₂₅₀₋₂₇₀ and A₂₂₀₋₂₄₀ of the eluate, d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the relative amount of empty capsids in the composition.

In some embodiments, the UV absorbance at about 250 nm to about 270 nm is a UV absorbance at about 260 nm. In some embodiments, the UV absorbance at about 220 nm to about 240 nm is a UV absorbance at about 230 nm.

In some embodiments, a PA260/PA230 ratio less than the PA260/PA230 ratio of a pure sample of full rAAV capsids is indicative of empty and/or partial capsids and in the composition. In some embodiments, a PA260/PA230 ratio equal to or less than a PA260/PA230 ratio of a rAAV sample with a known percentage of empty and/or partial capsids is indicative of empty and/or partial capsids in the composition. In some embodiments, the relative amount of empty capsids in the composition is determined by comparing the PA260/PA230 ratio of the eluate with a linear relationship between the PA260/PA230 ratio and the percent full capsids established with a plurality of rAAV preparations with different known percentages of full capsids. In some embodiments, the linear relationship between the PA260/PA230 ratio and the percent full capsids is determined by plotting the PA260/PA230 ratio versus the percent of full capsids of the plurality of rAAV preparations. In some embodiments, the linear relationship is represented by the equation % full capsids=(PA260/PA230−a)/b, wherein a is the PA260/PA230 of empty capsids and b is the slope of the linear equation determined from the plot of the PA260/PA230 ratio versus the percent of full capsids for the plurality of rAAV preparations. In some embodiments, the plurality of rAAV preparations with different known percentages of full capsids comprises three or more rAAV preparations wherein the three or more rAAV preparations comprise known ratios of full capsids to empty capsids ranging from 1:0 to 0:1. In some embodiments, the plurality of rAAV preparations comprise rAAV capsids of the same serotype as the rAAV particles in the composition. In some embodiments, the full capsids of the plurality of rAAV preparations comprise viral genomes that are at least about 90% in size compared to viral genomes of the rAAV particles in the composition. In some embodiments, the full capsids of the plurality of rAAV preparations comprise viral genomes that are the same as the viral genomes of the rAAV particles in the composition. In some embodiments, the plurality of rAAV preparations comprise capsids of the same serotype and the full capsids comprise the same viral genomes as the rAAV particles in the composition.

In some embodiments of the invention, the chromatography is size exclusion chromatography, ion exchange chromatography, affinity chromatography, mixed mode chromatography, hydrophobic interaction chromatography, or apatite chromatography. In some embodiments, the chromatography is a column chromatography. In some embodiments, the chromatography is high performance liquid chromatography (HPLC) or ultra-high performance liquid chromatography (UHPLC).

In some embodiments of the invention, the chromatography is size exclusion chromatography that utilizes a size exclusion chromatography column. In some embodiments, the size exclusion chromatography column comprises particles about 3 μm to about 10 μm in diameter. In some embodiments, the size exclusion chromatography column comprises particles about 5 μm in diameter. In some embodiments, the size exclusion chromatography column comprises particles with pores of about 100 Å to about 1000 Å in size. In some embodiments, the size exclusion chromatography column comprises particles with pores of about 500 Å in size. In some embodiments, the particles comprise silica. In some embodiments, the column has an internal diameter of between about 4.0 to about 7.8 mM and a length of between about 50 mm to about 300 mm, and in particular about 7.8 mm by about 300 mm, about 4.6 mm by about 100 mm, or about 4.6 mm by about 150 mm. In some embodiments, the size exclusion chromatography further utilizes a guard column. In some embodiments, the guard column comprises the same particles as the size exclusion chromatography column. In some embodiments, the guard column has an internal diameter of between about 4.0 to about 7.8 mM and a length of between about 30 mm to about 50 mm, and in particular is about 7.8 mm by about 50 mm, or about 4.6 by about 50 mm.

In some embodiments, a mobile phase of the size exclusion chromatography comprises phosphate buffered saline (PBS). In some embodiments, the PBS comprises about 100 mM to about 200 mM NaCl, about 1 mM to about 5 mM KCl, about 5 mM to about 20 mM Na₂HPO₄, and about 1 mM to about 3 mM KH₂PO₄. In some embodiments, the PBS comprises about 137 mM NaCl, about 2.7 mM KCl, about 10 mM Na₂HPO₄, and about 1.8 mM KH₂PO₄. In some embodiments, the PBS has a pH of about 6.5 to about 7.5 or about 7.0. In some embodiments, the chromatography is performed at a flow rate of about 0.5 mL/minute to about 1.0 mL/minute, about 0.7 mL/minute to about 0.8 mL/minute, or about 0.75 mL/minute. In some embodiments, the chromatography is performed at about 4° C. to about 35° C. In some embodiments, the chromatography is performed at about 25° C., about 30° C., or about 35° C. In some embodiments, about 5 μL to about 500 μL or about 10 μL to about 75 μL are subjected to the chromatography.

In some embodiments of the invention, the titer of rAAV in the composition is about 1×10¹¹ to about 1×10¹⁴ capsid particles/mL (cp/mL) or about 5×10¹² cp/mL. In some embodiments, the titer of rAAV in the composition is about 1×10¹⁰ to about 1×10¹⁴ viral genomes/mL (vg/mL). In some embodiments, greater than about 70% of the rAAV particles in the composition are full rAAV capsids. In some embodiments, greater than about 70% to about 95% of the rAAV particles in the composition are full rAAV capsids. In some embodiments, the rAAV particles in the composition have been purified using one or more purification steps. In some embodiments, the recombinant viral particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAV13 capsid, an AAV14 capsid, an AAV15 capsid, an AAV16 capsid, an AAVrh20 capsid, an AAV.rh39 capsid, an AAV.Rh74 capsid, an AAV.RHM4-1 capsid, an AAV.hu37 capsid, an AAV.Anc80 capsid, an AAV.Anc80L65 capsid, an AAV.PHP.B capsid, an AAV2.5 capsid, an AAV2tYF capsid, an AAV3B capsid, an AAV.LK03 capsid, an AAV.HSC1 capsid, an AAV.HSC2 capsid, an AAV.HSC3 capsid, an AAV.HSC4 capsid, an AAV.HSC5 capsid, an AAV.HSC6 capsid, an AAV.HSC7 capsid, an AAV.HSC8 capsid, an AAV.HSC9 capsid, an AAV.HSC10 capsid, an AAV.HSC11 capsid, an AAV.HSC12 capsid, an AAV.HSC13 capsid, an AAV.HSC14 capsid, an AAV.HSC15 capsid, an AAV.HSC16 capsid, an AAV2R471A capsid, an AAV2/2-7m8 capsid, an AAV DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid rAAV2/HBoV1 (chimeric AAV/human bocavirus virus 1). In some embodiments, the recombinant viral particle comprises an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, an AAV12 ITR, an AAV-13 ITR, an AAV-14 ITR, an AAV-15 ITR, an AAV-16 ITR, an AAV.rh20 ITR, an AAV.rh39 ITR, an AAV.rh74 ITR, an AAV.rhM4-1 ITR, an AAV.hu37 ITR, an AAV.Anc80 ITR, an AAV DJ ITR, a goat AAV ITR, a bovine AAV ITR, or a mouse AAV ITR. In some embodiments, the rAAV genome is 2500 bases to 5500 bases in length. In some embodiments, the recombinant viral particles comprise a self-complementary AAV (scAAV) genome.

In some embodiments, the rAAV particles are rAAV5 particles. In some embodiments, the rAAV particles are rAAV5 particles and the relative amount of empty capsids is calculated from the equation PA260/PA230=0.0057 (relative percent full capsids)+0.1137. In some embodiments, the rAAV particles are rAAV1 particles. In some embodiments, the rAAV particles are rAAV1 particles and the relative amount of empty capsids is calculated from the equation PA260/PA230=0.0054 (relative percent full capsids)+0.0886.

In some embodiments, the invention provides methods of monitoring the removal of empty capsids during the purification of a composition of rAAV particles, the method comprising collecting a sample of the composition before and following one or more steps of the purification process and analyzing each collected sample for the relative amount of empty capsids according to any of the methods described herein, wherein a decrease in the relative amount of empty capsids between the samples subsequently collected indicates removal of empty capsids from the preparation of rAAV particles. In some embodiments, the invention provides methods of monitoring the removal of empty capsids during the purification of a composition of rAAV particles, the method comprising: a) determining the relative amount of empty capsids according any of the methods described herein in a first sample collected before the purification process or before a step of the purification of the process, b) determining the relative amount of empty capsids according any one of methods described herein in a second sample collected following the purification process or a step of the purification of the process, wherein a decrease in the relative amount of empty capsids between the second and first collected samples indicates removal of empty capsids from the preparation of rAAV particles.

In some aspects, the invention provides kits for measuring the relative amount empty capsids in a composition of rAAV particles according the any of the methods described herein. In some embodiments, the kit comprises chromatography columns and/or buffers for use in the methods described herein. In some embodiments, the kit comprises three or more reference standards wherein the three or more reference standards comprise known ratios of full capsids to empty capsids ranging from 1:0 to 0:1.

### BRIEF DESCRIPTION OF THE DRAWINGS

FIGS. 1A-1C show the results of size-exclusion chromatography (SEC) of AAV5 samples using various columns. FIG. 1A shows SEC chromatograms of an AAV5 drug substance (DS) sample. The DS sample was diluted with Dulbecco's phosphate buffered saline (DPBS) to approximately 5.0×10¹² cp/mL and 25 μL of high performance liquid chromatography (HPLC) sample was applied onto the column. The elution was monitored with a photodiode array (PDA) detector. HPLC conditions: SEC-300 column (gray trace), SEC-500 column (black trace at 260 nm, dotted black trace at 280 nm), and SEC-1000 column (dashed gray trace). For SEC-300 and SEC-1000, only traces at 260 nm were shown for clarity. Mobile phase: DPBS; flow rate: 0.75 mL/min; column temperature: 25° C.; run time: 20 mins; detection at 260 nm and 280 nm. FIG. 1B shows chromatogram of a different lot of DS sample showing aggregated AAVs and monomer at 260 nm resolved by the SEC-500 column. The inset shows the high molecular weight species 1 (HMWS 1, 0.5% by peak area) and high molecular weight species 2 (HMWS 2, 1.7%). FIG. 1C shows the peak areas detected at 260 nm and 280 nm, showing a linear relationship with the number of capsids injected. PA260/PA280 remains constant across the range tested (5.0×10¹⁰ to 3.5×10¹¹ capsids, 10-75 μL injection of 5.0×10¹² cp/mL).

FIG. 2 shows predicted and measured A260/A280 ratios of the AAV samples containing various amounts of full capsids vs. the expected percent full capsids in the samples.

FIGS. 3A-3E show the results of SEC with dual-wavelength detection (SEC-DW) for the AAV spiked samples. FIG. 3A shows chromatograms of a series of AAV5 HPLC standards containing various percentages of full capsids at 260 nm. From front to back, the empty capsids, spiked samples with increased amount of full capsids from approximately 20% to 80%, and full capsids (91% full and 9% partial). FIG. 3B shows chromatograms of the empty, spiked samples and full capsids at 280 nm. FIG. 3C shows chromatograms of the empty, spiked samples, and full capsids at 230 nm. Chromatograms of one of the standard samples were not shown as the loading led to different absorbance intensities. The AAV capsids (50 μL per injection) were applied onto a size exclusion column (Sepax SRT® SEC-500, 5 μm, 500 Å, 7.8×300 mm) equipped with a guard column (SRT® SEC-500, 5 μm, 500 Å, 7.8×50 mm) equilibrated at 25° C. The capsids were eluted with Dulbecco's phosphate buffered saline, pH 7.0 at a flow rate of 0.75 mL/min. FIG. 3D shows the peak area ratios at 260 nm and 280 nm (PA260/PA280) and the peak area ratios at 260 nm and 230 nm (PA260/PA230) by SEC for the AAV5 samples. The curve fitting function changed from quadratic (for PA260/PA280) to linear regression (for PA260/PA230). FIG. 3E shows the PA260/PA230 ratio of the AAV5 drug substance (n=8), working reference standard (WRS, n=4), and empty capsids (n=6) samples obtained from multiple assay occasions performed on different days. The percent full capsids in the DS (n=8) and WRS (n=4) samples were calculated based on the linear relationship established between the PA260/PA230 and the percent full capsids (y=0.0057x+0.1137). The % RSD values are 0.58%, 0.54% and 5.30% for the PA260/PA230 values of the DS, WRS and empty capsids samples. The % RSD values are 0.73% and 0.67% for the percent full capsids of the DS and WRS samples, respectively.

FIG. 4 shows the peak area at 260 nm (PA260) and the peak area at 230 nm (PA230) relative to the number of capsids applied onto the column.

FIGS. SA-SD show representative sedimentation velocity analytical ultracentrifugation (AUC-SV) data shown as sedimentation coefficient distribution plots. FIG. 5A shows empty capsids. FIG. 5B shows spiked sample 3 (see Table 3). FIG. 5C shows spiked sample 5 (see Table 3). FIG. 5D shows full capsids. The sedimentation of AAV particles was monitored at 260 nm. The raw data were fitted with the Lamm equation using the c(s) model in SEDFIT. The x axis represents the sedimentation coefficient in Svedberg unit S, and they axis represents the concentration as a function of sedimentation coefficient.

FIGS. 6A-6B show representative cryogenic electron microscopy (Cryo-EM) images of two spiked samples. FIG. 6A shows an image of the spiked sample 3 containing 54% full capsids by Cryo-EM. FIG. 6B Image of the full capsid sample containing 97% full capsids by Cryo-EM. An empty capsid and a full capsid are indicated in Image A. The cross central sections of the empty and full capsids show lack of and significant internal density, respectively.

FIGS. 7A-7B show the percentage of full capsids in spiked samples as determined by SEC-DW, AUC-SV and Cryo-EM. FIG. 7A shows the percent full capsids in the spiked samples obtained from SEC-DW, AUC-SV, and Cryo-EM. The results were plotted against the expected percent full capsids and fitted with linear regressions. The R² values are 0.997, 0.993 and 0.993 for SEC-DW, AUC-SV, and Cryo-EM. The slope values are 0.9892 for SEC-DW, 1.0623 for AUC-SV, and 1.0378 for Cryo-EM. FIG. 7B shows the results from SEC-DW, AUC-SV, and Cryo-EM are presented as a bar graph to visualize the direct comparison.

FIG. 8 shows absorbance spectra for an empty capsids sample and a full capsids sample. The absorbance spectra (220 nm-400 nm) were extracted from the SE-HPLC data acquired.

FIGS. 9A-9D show SEC-DW results of a second AAV DS candidate. FIG. 9A show SEC chromatograms of the empty capsids of a different AAV5 drug substance sample (candidate 2) at 230 nm and 260 nm. FIG. 9B shows SEC chromatograms of the DS sample of the AAV5 drug candidate 2 at 230 nm and 260 nm. FIG. 9C shows extracted absorbance spectra of the empty capsids and the DS sample (candidate 2). FIG. 9D shows the PA260 and PA230 show excellent linearity with the number of capsids (different injection volumes from the same samples) applied onto the column.

FIG. 10 shows the peak area ratios at 260 nm and 280 nm (PA260/PA280) and the peak area ratios at 260 nm and 230 nm (PA260/PA230) by SEC for the AAV1 samples. The curve fitting function changed from quadratic (for PA260/PA280) to linear regression (for PA260/PA230).

### DETAILED DESCRIPTION

In some aspects, the invention provides methods to determine the presence of empty and/or partial capsids in a composition comprising recombinant adeno-associated virus (rAAV) particles, the method comprising: a) subjecting the composition to chromatography under conditions to separate the rAAV particles from impurities in the composition in an eluate of the chromatography; b) measuring the UV absorbance at about 250 nm to about 270 nm (A₂₅₀₋₂₇₀) and about 220 nm to about 240 nM (A₂₂₀₋₂₄₀) of the eluate, c) plotting chromatograms of the A₂₅₀₋₂₇₀ and A₂₂₀₋₂₄₀ of the eluate, d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the presence of empty and/or partial capsids in the composition.

In some aspects, the invention provides methods of measuring the relative amount of empty and/or partial capsids in a composition comprising rAAV particles, the method comprising: a) subjecting the composition to chromatography under conditions to separate the rAAV particles from impurities in the composition in an eluate of the chromatography; b) measuring the UV absorbance at about 250 nm to about 270 nm (A₂₅₀₋₂₇₀) and about 220 nm to about 240 nM (A₂₂₀₋₂₄₀) of the eluate, c) plotting chromatograms of the A₂₂₀₋₂₇₀ and A₂₂₀₋₂₄₀ of the eluate, d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the relative amount of empty capsids in the composition.

### General Techniques

The techniques and procedures described or referenced herein are generally well understood and commonly employed using conventional methodology by those skilled in the art, such as, for example, the widely utilized methodologies described in Molecular Cloning: A Laboratory Manual(Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and issue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011).

### Definitions

A “vector,” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two inverted terminal repeat sequences (ITRs).

A “recombinant AAV vector (recombinant adeno-associated viral vector)” refers to a polynucleotide vector comprising one or more heterologous sequences (i.e., nucleic acid sequence not of AAV origin) that are flanked by at least one AAV inverted terminal repeat sequences (ITR). In some embodiments, the recombinant nucleic acid is flanked by two inverted terminal repeat sequences (ITRs). Such recombinant viral vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of AAV packaging functions and suitable helper functions. A recombinant viral vector can be in any of a number of forms, including, but not limited to, plasmids, linear artificial chromosomes, complexed with lipids, encapsulated within liposomes, and encapsidated in a viral particle, for example, an AAV particle. A recombinant viral vector can be packaged into an AAV virus capsid to generate a “recombinant adeno-associated viral particle (recombinant viral particle)”.

An “rAAV virus” or “rAAV viral particle” refers to a viral particle composed of at least one AAV capsid protein and an encapsidated rAAV vector genome as defined above.

As used herein, an “empty AAV capsid” is an AAV capsid in which essentially no AAV nucleic acid has been encapsidated. As used herein, a “partial AAV capsid” is an AAV capsid that has encapsidated an incomplete AAV genome (e.g., a truncated AAV genome, a fragment of an AAV genome, or an AAV genome with internal deletion(s)). In some embodiments, an incomplete AAV genome comprises or consists of less than about 90%, 80%, 70%, 60%, 50%, 40%, 30%, or 25% in length of the AAV genome of a full AAV capsid (capsid (i.e., harboring full-length AAV genome). In some embodiments, an incomplete AAV genome comprises or consists of less than about 70%, 60%, 50%, 40%, 30% or 25% in length of the AAV genome of a full AAV capsid. The percentage is calculated based on the full alignment of the two AAV genome sequences (with the full-length AAV genome being the longest sequence.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as siRNA.

The terms “genome particles (gp),” “genome equivalents,” or “genome copies” as used in reference to a viral titer, refer to the number of virions containing the recombinant viral DNA genome or RNA genome, regardless of infectivity or functionality. The number of genome particles in a particular vector preparation can be measured by procedures such as described in, for example, in Clark et al. (1999) Hum. Gene Ther., 10:1031-1039; Veldwijk et al. (2002) Mol. Ther., 6:272-278.

The terms “infection unit (iu),” “infectious particle,” or “replication unit,” as used in reference to a viral titer, refer to the number of infectious and replication-competent recombinant viral vector particles as measured by the infectious center assay, also known as replication center assay, as described, for example with AAV, in McLaughlin et al. (1988) J. Virol., 62:1963-1973.

The term “transducing unit (tu)” as used in reference to a viral titer, refers to the number of infectious recombinant viral vector particles that result in the production of a functional transgene product as measured in functional assays such as described in, for example, Xiao et al. (1997) Exp. Neurobiol., 144:113-124; or in Fisher et al. (1996) J. Virol., 70:520-532 (LFU assay).

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A helper virus provides “helper functions” which allow for the replication of AAV. A number of such helper viruses have been identified, including adenoviruses, herpesviruses, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions. Baculoviruses available from depositories include Autographa californica nuclear polyhedrosis virus.

A preparation or composition of rAAV is said to be “substantially free” of helper virus if the ratio of infectious AAV particles to infectious helper virus particles is at least about 10²:1; at least about 10⁴:1, at least about 10⁶:1; or at least about 10⁸:1 or more. In some embodiments, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

To “reduce” is to decrease, reduce or arrest an activity, function, and/or amount as compared to a reference. In certain embodiments, by “reduce” is meant the ability to cause an overall decrease of 20% or greater. In certain embodiments, by “reduce” is meant the ability to cause an overall decrease of 30% or greater. In certain embodiments, by “reduce” is meant the ability to cause an overall decrease of 40% or greater. In another embodiment, by “reduce” is meant the ability to cause an overall decrease of 50% or greater. In yet another embodiment, by “reduce” is meant the ability to cause an overall decrease of 75%, 85%, 90%, 95%, or greater.

A “reference” as used herein, refers to any sample, standard, or level that is used for comparison purposes. For example, when measuring the relative amount of empty capsids in a composition of rAAV particles, the amount of empty capsids may be compared to the amount of AAV particles in a preparation comprising known ratios of full capsids to empty capsids. In some embodiments, when monitoring the removal of empty capsids during the purification of a composition of rAAV particles, the purified AAV produced is compared to preparations comprising known ratios of full capsids to empty capsids. In other examples, a reference may refer to a standard procedure known in the art.

An “isolated” molecule (e.g., nucleic acid or protein) or cell means it has been identified and separated and/or recovered from a component of its natural environment. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) present in a solution, or by infectivity, or it can be measured in relation to a second, potentially interfering substance present in the source mixture, such as contaminants, including production culture contaminants or in-process contaminants, including helper virus, media components, and the like, as defined below.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise. For example, the phrase “a rAAV particle” includes one or more rAAV particles.

It is understood that aspects and embodiments of the invention described herein include “comprising,” “consisting,” and/or “consisting essentially of” aspects and embodiments.

Methods to Detect and Quantify Empty rAAV Capsids

### Dual Wavelength Detection

In some aspects, the invention provides methods to determine the presence of empty and/or partial capsids in a composition comprising recombinant adeno-associated virus (rAAV) particles, the method comprising: a) subjecting the composition to chromatography under conditions to separate the rAAV particles from impurities in the composition in an eluate of the chromatography; b) measuring the UV absorbance at about 250 nm to about 270 nm (A₂₅₀₋₂₇₀) and about 220 nm to about 240 nM (A₂₂₀₋₂₄₀) of the eluate, c) plotting chromatograms of the A₂₅₀₋₂₇₀ and A₂₂₀₋₂₄₀ of the eluate, d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the presence of empty and/or partial capsids in the composition.

As used herein, the term “PA260/PA230” refers to the ratio of peak area of any plot of UV absorbance of the eluate at between about A₂₅₀ and A₂₇₀ (e.g., any of 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, or 270 nm) and the peak area of any plot of UV absorbance of the eluate at between about A₂₂₀ and A₂₄₀ (e.g., any of 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, or 240 nm).

In some aspects, the invention provides methods of measuring the relative amount empty and/or partial capsids in a composition comprising rAAV particles, the method comprising: a) subjecting the composition to chromatography under conditions to separate the rAAV particles from impurities in the composition in an eluate of the chromatography; b) measuring the UV absorbance at about 250 nm to about 270 nm (A₂₅₀₋₂₇₀) and about 220 nm to about 240 nM (A₂₂₀₋₂₄₀) of the eluate, c) plotting chromatograms of the A₂₅₀₋₂₇₀ and A₂₂₀₋₂₄₀ of the eluate, d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the relative amount of empty capsids in the composition.

In some embodiments, the UV absorbance at about 250 nm to about 270 nm is a UV absorbance at any wavelength between about 250 nm and 270 nm. In some embodiments, the UV absorbance at about 250 nm to about 270 nm is a UV absorbance at 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, or 270 nm. In some embodiments, the UV absorbance at about 250 nm to about 270 nm is a UV absorbance at any wavelength between about 255 nm and 265 nm. In some embodiments, the UV absorbance at about 250 nm to about 270 nm is a UV absorbance at about 260 nm.

In some embodiments, the UV absorbance at about 220 nm to about 240 nm is a UV absorbance at any wavelength between about 220 nm and 240 nm. In some embodiments, the UV absorbance at about 220 nm to about 230 nm is a UV absorbance at 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, or 240 nm. In some embodiments, the UV absorbance at about 220 nm to about 240 nm is a UV absorbance at any wavelength between about 225 nm and 235 nm. In some embodiments, the UV absorbance at about 220 nm to about 240 nm is a UV absorbance at about 230 nm.

In some embodiments, a PA260/PA230 ratio less than the PA260/PA230 ratio of a pure sample of full rAAV capsids is indicative of empty and/or partial capsids and in the composition. As demonstrated in the Examples, other methods for determining the relative amounts of full and empty AAV capsids are known; for example, analytical ultracentrifugation and cryo-electron microscopy. An example of analytic ultracentrifugation is provided by WO 2016/118520.

In some embodiments, the relative amount of empty capsids in the composition is determined by comparing the PA260/PA230 ratio of the eluate with a linear relationship between the PA260/PA230 ratio and the percent full capsids established with a plurality of rAAV preparations with different known percentages of full capsids. In some embodiments, the linear relationship between the PA260/PA230 ratio and the percent full capsids is determined by plotting the PA260/PA230 ratio versus the percent of full capsids of the plurality of rAAV preparations. In some embodiments, curve fitting is used to establish the relationship between the PA260/PA230 ratio and the percent full capsids. In some embodiments, curve fitting is used to generate the equation

% full capsids=(PA260/PA230−a)/b

wherein a is the PA260/PA230 of empty capsids and b is the slope of the linear equation determined from the plot of the PA260/PA230 ratio versus the percent of full capsids for the plurality of rAAV preparations. In some embodiments, the rAAV particles are rAAV5 particles and the relative amount of empty capsids is calculated from the equation PA260/PA230=0.0057 (relative percent full capsids)+0.1137. In some embodiments, the rAAV particles are rAAV1 particles and the relative amount of empty capsids is calculated from the equation PA260/PA230=0.0054 (relative percent full capsids)+0.0886. In some embodiments, curve fitting demonstrates a linear relationship with a coefficient of determination, R², greater than 0.9, 0.95, 0.96, 0.97, 0.98, 0.99, 0.991, 0.992, 0.993, 0.994, or 0.995.

In some embodiments, the plurality of rAAV preparations with different known percentages of full capsids comprises three or more rAAV preparations wherein the three or more rAAV preparations comprise known ratios of full capsids to empty capsids ranging from 1:0 to 0:1. In some embodiments, the three or more rAAV preparations comprise known ratios of full capsids to empty capsids of any one of 1:0, 0.9:0.1, 0.8:0.2, 0.7:0.3, 0.6:0:4, 0.5:0.5, 0.4:0.6, 0.3:0.7, 0.2:0.8, 0.1:0.9, and 0:1. In some embodiments, the three or more rAAV preparations include preparations with low levels of empty capsids (e.g., >75% full capsids), approximately equal amounts of full and empty capsids (e.g., ˜50% full capsids), and high levels of empty capsids (e.g., <25% full capsids). In some embodiments, the plurality of rAAV preparations comprise rAAV capsids of the same serotype as the rAAV particles in the composition. In some embodiments, the full capsids of the plurality of rAAV preparations comprise viral genomes that are at least about 90% in size (e.g., in length) compared to viral genomes of the rAAV particles in the composition. In some embodiments, the full capsids of the plurality of rAAV preparations comprise viral genomes that are about any of 90%, 95%, 100%, 105% or 110% in size (e.g., in length) compared to viral genomes of the rAAV particles in the composition. The percentage is calculated based on the full alignment of the two AAV genome sequences. In some embodiments, the full capsids of the plurality of rAAV preparations comprise viral genomes that are essentially the same size compared to viral genomes of the rAAV particles in the composition. In some embodiments, the full capsids of the plurality of rAAV preparations comprise viral genomes that are the same as the viral genomes of the rAAV particles in the composition. In some embodiments, the full capsids of the plurality of rAAV preparations comprise viral genomes that are the different as the viral genomes of the rAAV particles in the composition, provided that the viral genomes are at least about 90% in size (e.g., in length) compared to viral genomes of the rAAV particles in the composition (as described above). In some embodiments, the full capsids of the plurality of rAAV preparations comprise the same serotype capsids and the full capsids comprise the same viral genomes as the rAAV particles in the composition.

### Chromatography

In some embodiments, the invention provides methods of measuring the relative amount of empty and/or partial capsids in a composition comprising rAAV particles. The methods use chromatography to separate impurities from the rAAV capsids (e.g., full, empty and partial capsids). Any chromatography methods that separates rAAV capsids from impurities are contemplated. In some embodiments, full, empty and partial capsids elute together. Non-limiting impurities may include high molecular weight species, host cell proteins, and helper viral proteins.

In some embodiments, chromatography used to measure the relative amount empty and/or partial capsids in a composition comprising rAAV particles is size exclusion chromatography, ion exchange chromatography, affinity chromatography, mixed mode chromatography, hydrophobic interaction chromatography, or apatite chromatography. In some embodiments, the chromatography utilizes column chromatography. In some embodiments, the chromatography is high performance liquid chromatography (HPLC) or ultra-high performance liquid chromatography (UHPLC).

In some embodiments, the chromatography is an ion exchange chromatography; for example, an anion exchange chromatography or a cation exchange chromatography. In some embodiments, the anion exchange chromatography material is a solid phase that is positively charged and has free anions for exchange with anions in an aqueous solution passed over or through the solid phase. In some embodiments, the anion exchange material may comprise a primary amine, a secondary amine, a tertiary amine or a quaternary ammonium ion functional group, a polyamine functional group, or a diethylaminoaethyl functional group. In some embodiments, the cation exchange chromatography material is a solid phase that is positively charged and has free cations for exchange with cations in an aqueous solution passed over or through the solid phase. The cation exchange material may comprise a carboxylic acid functional group or a sulfonic acid functional group such as, but not limited to, sulfonate, carboxylic, carboxymethyl sulfonic acid, sulfoisobutyl, sulfoethyl, carboxyl, sulphopropyl, sulphonyl, sulphoxyethyl, or orthophosphate.

In some embodiments, the chromatography is an affinity chromatography in which the chromatography material comprises a ligand that specifically binds AAV (e.g., an antibody).

In some embodiments, chromatography is a mixed-mode chromatography. In some embodiments, the mixed-mode chromatography material comprises functional groups capable of one of more of the following functionalities: anionic exchange, cation exchange, hydrogen bonding, and hydrophobic interactions. In some embodiments, the mixed-mode material comprises functional groups capable of anionic exchange and hydrophobic interactions. In some embodiments, the mixed-mode material comprises functional groups capable of cationic exchange and hydrophobic interactions.

In some embodiments, the chromatography is hydrophobic interaction chromatography (HIC) chromatography. Hydrophobic interaction chromatography is a liquid chromatography technique that separates biomolecules according to hydrophobicity.

In some embodiments, the chromatography is hydroxyapatite chromatography. In some embodiments, the hydroxyapatite comprises (Ca₅(PO₄)₃OH)₂. In some embodiments, the hydroxyapatite chromatography is ceramic hydroxyapatite chromatography. In some embodiments, the hydroxyapatite chromatography is CHT ceramic hydroxyapatite chromatography. Examples of hydroxyapatite materials are known in the art include, but are not limited to CHT ceramic hydroxyapatite, Type I and Type II.

In some embodiments, the chromatography is size exclusion chromatography. In some embodiments, the invention provides methods of measuring the relative amount of empty and/or partial capsids in a composition comprising rAAV particles, the method comprising: a) subjecting the composition to size exclusion chromatography under conditions to separate the rAAV particles from impurities in the composition in an eluate of the chromatography; b) measuring the UV absorbance at about 250 nm to about 270 nm (A₂₅₀₋₂₇₀) and about 220 nm to about 240 nM (A₂₂₀₋₂₄₀) of the eluate, c) plotting chromatograms of the A₂₅₀₋₂₇₀ and A₂₂₀₋₂₄₀ of the eluate, d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the relative amount of empty capsids in the composition. In some embodiments, the size exclusion chromatography is a liquid chromatography technique that separates biomolecules according to their size, shape, hydrodynamic radius and/or volume. Size exclusion chromatography materials come in different particle sizes and pore sizes for efficient separation of molecules of particular weight ranges. In some embodiments, the size exclusion chromatography materials comprise silica. In some embodiments, the size exclusion chromatography utilizes a size exclusion chromatography column.

In some embodiments, the size exclusion chromatography column comprises particles about 1 μm to about 10 μm in diameter. In some embodiments, the size exclusion chromatography comprises particles of any of about 1 μm, about 2 μm, about 3 μm, about 4 μm, about 5 μm, about 6 μm, about 7 μm, about 8 μm, about 9 μm, about 10 μm, or more than about 10 μm in diameter. In some embodiments, the size exclusion chromatography comprises particles between about any of 1 μm and 10 μm, 1 μm and 9 μm, 1 μm and 8 μm, 1 μm and 7 μm, 1 μm and 6 μm, 1 μm and 5 μm, 1 μm and 4 μm, 1 μm and 3 μm, 1 μm and 2 μm, 2 μm and 10 μm, 2 μm and 9 μm, 2 μm and 8 μm, 2 μm and 7 μm, 2 μm and 6 μm, 2 μm and 5 μm, 2 μm and 4 μm, 2 μm and 3 μm, 3 μm and 10 μm, 3 μm and 9 μm, 3 μm and 8 μm, 3 μm and 7 μm, 3 μm and 6 μm, 3 μm and 5 μm, 3 μm and 4 μm, 4 μm and 10 μm, 4 μm and 9 μm, 4 μm and 8 μm, 4 μm and 7 μm, 4 μm and 6 μm, 4 μm and 5 μm, 5 μm and 10 μm, 5 μm and 9 μm, 5 μm and 8 μm, 5 μm and 7 μm, 5 μm and 6 μm, 6 μm and 10 μm, 6 μm and 9 μm, 6 μm and 8 μm, 6 μm and 7 μm, 7 μm and 10 μm, 7 μm and 9 μm, 7 μm and 8 μm, 8 μm and 10 μm, 8 μm and 9 μm, or 9 μm and 10 μm.

In some embodiments, the size exclusion chromatography column comprises particles with pores of about 100 Å to about 1000 Å in size. In some embodiments, the size exclusion chromatography column comprises particles with pores of any of about 100 Å, about 200 Å, about 300 Å, about 400 Å, about 500 Å, about 600 Å, about 700 Å, about 800 Å, about 900 Å, or about 1000 Å. In some embodiments, the size exclusion chromatography column comprises particles with pores of between any of about 100 Å and about 1000 Å, about 100 Å and about 900 Å, about 100 Å and about 800 Å, about 100 Å and about 700 Å, about 100 Å and about 600 Å, about 100 Å and about 500 Å, about 100 Å and about 400 Å, about 100 Å and about 300 Å, about 100 Å and about 200 Å, about 200 Å and about 1000 Å, about 200 Å and about 900 Å, about 200 Å and about 800 Å, about 200 Å and about 700 Å, about 200 Å and about 600 Å, about 200 Å and about 500 Å, about 200 Å and about 400 Å, about 200 Å and about 300 Å, about 300 Å and about 1000 Å, about 300 Å and about 900 Å, about 300 Å and about 800 Å, about 300 Å and about 700 Å, about 300 Å and about 600 Å, about 300 Å and about 500 Å, about 300 Å and about 400 Å, about 400 Å and about 1000 Å, about 400 Å and about 900 Å, about 400 Å and about 800 Å, about 400 Å and about 700 Å, about 400 Å and about 600 Å, about 400 Å and about 500 Å, about 500 Å and about 1000 Å, about 500 Å and about 900 Å, about 500 Å and about 800 Å, about 500 Å and about 700 Å, about 500 Å and about 600 Å, about 600 Å and about 1000 Å, about 600 Å and about 900 Å, about 600 Å and about 800 Å, about 600 Å and about 700 Å, about 700 Å and about 1000 Å, about 700 Å and about 900 Å, about 700 Å and about 800 Å, about 800 Å and about 1000 Å, about 800 Å and about 900 Å, or about 900 Å and about 1000 Å.

In some embodiments, the size exclusion chromatography column has an internal diameter of between about 4.0 to about 7.8 mM and a length of between about 50 mm to about 300 mm. In some embodiments, the size exclusion chromatography column has an internal diameter of about 4.0 mm, 4.6 mm or 7.8 mm. In some embodiments, the size exclusion chromatography column has a length of about 50 mm, 100 mm, 150 mm or 300 mm. In some embodiment, the size exclusion chromatography column is about 7.8 mm by about 300 mm, about 4.6 mm by about 100 mm, or about 4.6 mm by about 150 mm.

In some embodiments, the size exclusion chromatography further utilizes a guard column. In some embodiments, the guard column comprises the same particles as the size exclusion chromatography column; e.g., particles made of the same material as the size exclusion chromatography column, with particles of the same size as the size exclusion chromatography column, and with pores of the same size as the pores in the size exclusion chromatography column.

In some embodiments, the guard column has an internal diameter of between about 4.0 to about 7.8 mM and a length of between about 30 mm to about 50 mm. In some embodiments, the guard column has an internal diameter of about 4.0 mm, 4.6 mm or 7.8 mm. In some embodiments, the guard column has a length of about 30 mm, 40 mm, or 50 mm. In some embodiment, the guard column is about 7.8 mm by about 50 mm.

In some embodiments, the chromatography utilizes a mobile phase. In some embodiments, the mobile phase is an aqueous mobile phase. In some embodiments, the aqueous phase is a salt solution (e.g., an aqueous salt solution). In some embodiments, the mobile phase of the chromatography comprises phosphate buffered saline (PBS). In some embodiments, the mobile phase of the size exclusion chromatography comprises PBS. PBS is a common buffered salt solution known in the art. In some embodiments, the PBS comprises about 100 mM to about 200 mM NaCl, about 1 mM to about 5 mM KCl, about 5 mM to about 20 mM Na₂HPO₄, and about 1 mM to about 3 mM KH₂PO₄. In some embodiments, the PBS comprises about 137 mM NaCl, about 2.7 mM KCl, about 10 mM Na₂HPO₄, and about 1.8 mM KH₂PO₄.

In some embodiments, the mobile phase of the chromatography has a pH of about 6.0 to about 8.0. In some embodiments, the pH of the mobile phase is any of about 6.0, 6.5, 7.0, 7.5 or 8.0. In some embodiments, the pH of the mobile phase is between about any of 6.0 and 8.0, 6.5 and 8.0, 7.0 and 8.0, 7.5 and 8.0, 6.0 and 7.5, 6.5 and 7.5, 7.0 and 7.5, 6.0 and 7.0, 6.5 and 7.0, or 6.0 and 6.5. In some embodiments, the mobile phase of the chromatography is PBS at a pH of about 6.0 to about 8.0. In some embodiments, the mobile phase of the size exclusion chromatography is PBS at a pH of about 6.0 to about 8.0. In some embodiments, the mobile phase of the size exclusion chromatography is PBS at a pH of about 7.0.

In some embodiments, the chromatography (e.g., size exclusion chromatography) is performed at a flow rate of about 0.3 mL/minute to about 1.0 mL/minute. In some embodiments, the chromatography is performed at a flow rate of any of about 0.3 mL/minute, about 0.5 mL/minute, 0.6 mL/minute, 0.7 mL/minute, 0.75 mL/minute, 0.8 mL/minute, 0.9 mL/minute, or 1.0 mL/minute. In some embodiments, the chromatography is performed at a flow rate between any of about 0.3 mL/minute and 1.0 mL/minute, about 0.5 mL/minute and 1.0 mL/minute, about 0.6 mL/minute and 1.0 mL/minute, about 0.7 mL/minute and 1.0 mL/minute, about 0.75 mL/minute and 1.0 mL/minute, about 0.8 mL/minute and 1.0 mL/minute, about 0.9 mL/minute and 1.0 mL/minute, about 0.3 mL/minute and 0.9 mL/minute, about 0.5 mL/minute and 0.9 mL/minute, about 0.6 mL/minute and 0.9 mL/minute, about 0.7 mL/minute and 0.9 mL/minute, about 0.75 mL/minute and 0.9 mL/minute, about 0.8 mL/minute and 0.9 mL/minute, about 0.3 mL/minute and 0.8 mL/minute, about 0.5 mL/minute and 0.8 mL/minute, about 0.6 mL/minute and 0.8 mL/minute, about 0.7 mL/minute and 0.8 mL/minute, about 0.75 mL/minute and 0.8 mL/minute, about 0.3 mL/minute and 0.75 mL/minute, about 0.5 mL/minute and 0.75 mL/minute, about 0.6 mL/minute and 0.75 mL/minute, about 0.7 mL/minute and 0.75 mL/minute, about 0.3 mL/minute and 0.7 mL/minute, about 0.5 mL/minute and 0.7 mL/minute, about 0.3 mL/minute and 0.6 mL/minute, about 0.5 mL/minute and 0.6 mL/minute or about 0.3 mL/minute and 0.5 mL/minute. In some embodiments, the size exclusion chromatography is performed at a flow rate of any of about 0.75 mL/minute.

In some embodiments, the chromatography (e.g., size exclusion chromatography) is performed at about 4° C. to about 35° C. In some embodiments, the chromatography is performed at about 4° C., about 20° C., about 25° C., about 30° C., or about 35° C. In some embodiments, the chromatography is performed as between any of about 4° C. to about 35° C., about 20° C. to about 35° C., about 25° C. to about 35° C., about 30° C. to about 35° C., 4° C. to about 30° C., about 20° C. to about 30° C., about 25° C. to about 30° C., 4° C. to about 25° C., or about 20° C. to about 25° C. In some embodiments, the chromatography is performed at room temperature. In some embodiments, the size exclusion chromatography is performed at about 25° C.

In some embodiments, about 5 μL to about 500 μL of the composition comprising rAAV particles is subjected to chromatography (e.g., size exclusion chromatography). In some embodiments, about 5 μL, about 25 μL, about 50 μL, about 75 μL, about 100 μL, about 200 μL, about 300 μL, about 400 μL, or about 500 μL of the composition comprising rAAV particles is subjected to chromatography. In some embodiments, between any of about 5 μL and about 100 μL, about 25 μL and about 100 μL, about 50 μL and about 100 μL, about 75 μL and about 100 μL, 5 μL and about 75 μL, about 25 μL and about 75 μL, about 50 μL and about 75 μL, 5 μL and about 50 μL, about 25 μL and about 50 μL, or about 5 μL and about 25 μL of the composition comprising rAAV particles is subjected to chromatography. In some embodiments, about 50 μL of the composition comprising rAAV particles is subjected to size exclusion chromatography.

In some embodiments, the titer of rAAV in the composition subjected to chromatography (e.g., size exclusion chromatography) is about 1×10¹¹ to about 1×10¹⁴ capsid particles/mL (cp/mL). In some embodiments, the titer of rAAV in the composition subjected to chromatography between any of about 1×10¹¹ to about 1×10¹⁴ cp/mL, 5×10¹¹ to about 1×10¹⁴ cp/mL, 1×10¹² to about 1×10¹⁴ cp/mL, 5×10¹² to about 1×10¹⁴ cp/mL, 1×10¹³ to about 1×10¹⁴ cp/mL, 5×10¹³ to about 1×10¹⁴ cp/mL, 1×10¹¹ to about 5×10¹³ cp/mL, 5×10¹¹ to about 5×10¹³ cp/mL, 1×10¹² to about 5×10¹³ cp/mL, 5×10¹² to about 5×10¹³ cp/mL, 1×10¹³ to about 5×10¹³ cp/mL, 1×10¹¹ to about 1×10¹³ cp/mL, 5×10¹¹ to about 1×10¹³ cp/mL, 1×10¹² to about 1×10¹³ cp/mL, 5×10¹² to about 1×10¹³ cp/mL, 1×10¹¹ to about 5×10¹² cp/mL, 5×10¹¹ to about 5×10¹² cp/mL, 1×10¹² to about 5×10¹² cp/mL, 1×10¹¹ to about 1×10¹² cp/mL, 5×10¹¹ to about 1×10¹² cp/mL, or 1×10¹¹ to about 5×10¹¹ cp/mL. In some embodiments, the titer of rAAV in the composition subjected to size exclusion chromatography is about 5×10¹² cp/mL.

In some embodiments, the titer of rAAV in the composition subjected to chromatography (e.g., size exclusion chromatography) is about 1×10¹⁰ to about 1×10¹⁴ vector genomes/mL (vg/mL). In some embodiments, the titer of rAAV in the composition subjected to chromatography between any of about 1×10¹⁰ to about 1×10³ vg/mL, 5×10¹⁰ to about 1×10¹³ vg/mL, 1×10¹¹ to about 1×10³ vg/mL, 5×10¹¹ to about 1×10¹³ vg/mL, 1×10¹² to about 1×10¹³ vg/mL, 5×10¹² to about 1×10¹³ vg/mL, 1×10¹⁰ to about 5×10¹² vg/mL, 5×10¹⁰ to about 5×10¹² vg/mL, 1×10¹¹ to about 5×10¹² vg/mL, 5×10¹¹ to about 5×10¹² vg/mL, 1×10¹² to about 5×10¹² vg/mL, 1×10¹⁰ to about 1×10¹² vg/mL, 5×10¹⁰ to about 1×10¹² vg/mL, 1×10¹¹ to about 1×10¹² vg/mL, 5×10¹¹ to about 1×10¹² vg/mL, 1×10¹⁰ to about 5×10¹¹ vg/mL, 5×10¹⁰ to about 5×10¹¹ vg/mL, 1×10¹¹ to about 5×10¹¹ vg/mL, 1×10¹⁰ to about 1×10¹¹ vg/mL, 5×10¹⁰ to about 1×10¹¹ vg/mL, or 1×10¹⁰ to about 5×10¹⁰ vg/mL. In some embodiments, the titer of rAAV in the composition subjected to size exclusion chromatography is about 5×10¹² vg/mL.

In some embodiments, greater than about 70%, about 75%, about 80%, about 85%, about 90%, about 95%, or about 99%/o of the rAAV particles in the composition are full rAAV capsids. In some embodiments, between about any of 70% to 99%, 70% to 90%, 70% to 85%, 70% to 80%, 70% to 75%, 75% to 99%, 75% to 90%, 75% to 85%, 75% to 80%, 80% to 99%, 80% to 90%, 80% to 85%, 85% to 99%, 85% to 90%, or 90% to 99% of the rAAV particles in the composition are full rAAV capsids.

In embodiments of the embodiments described above, the rAAV particles comprise an AAV1 capsid, an AAV2 capsid, an AAV3 capsid, an AAV4 capsid, an AAV5 capsid, an AAV6 capsid (e.g., a wild-type AAV6 capsid, or a variant AAV6 capsid such as ShH10, as described in U.S. PG Pub. 2012/0164106), an AAV7 capsid, an AAV8 capsid, an AAVrh8 capsid, an AAVrh8R, an AAV9 capsid (e.g., a wild-type AAV9 capsid, or a modified AAV9 capsid as described in U.S. PG Pub. 2013/0323226), an AAV10 capsid, an AAVrh10 capsid, an AAV11 capsid, an AAV12 capsid, an AAV13 capsid, an AAV14 capsid, an AAV15 capsid, an AAV16 capsid, an AAVrh20 capsid, an AAV.rh39 capsid, an AAV.Rh74 capsid, an AAV.RHM4-1 capsid, an AAV.hu37 capsid, an AAV.Anc80 capsid, an AAV.Anc80L65 capsid, an AAV.PHP.B capsid, an AAV2.5 capsid, an AAV2tYF capsid, an AAV3B capsid, an AAV.LK03 capsid, an AAV.HSC1 capsid, an AAV.HSC2 capsid, an AAV.HSC3 capsid, an AAV.HSC4 capsid, an AAV.HSC5 capsid, an AAV.HSC6 capsid, an AAV.HSC7 capsid, an AAV.HSC8 capsid, an AAV.HSC9 capsid, an AAV.HSC1O capsid, an AAV.HSC11 capsid, an AAV.HSC12 capsid, an AAV.HSC13 capsid, an AAV.HSC14 capsid, an AAV.HSC15 capsid, an AAV.HSC16 capsid, a tyrosine capsid mutant, a heparin binding capsid mutant, an AAV2R471A capsid, an AAVAAV2/2-7m8 capsid, an AAV DJ capsid (e.g., an AAV-DJ/8 capsid, an AAV-DJ/9 capsid, or any other of the capsids described in U.S. PG Pub. 2012/0066783), an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid, or an AAV capsid described in U.S. Pat. No. 8,283,151 or International Publication No. WO/2003/042397. In embodiments of the embodiments described above, the rAAV particles comprise an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAV2R471A capsid, an AAV2/2-7m8 capsid, an AAV DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid rAAV2/HBoV1 (chimeric AAV/human bocavirus virus 1). In embodiments of the embodiments described above, the rAAV particles comprise a capsid which is selected from the group consisting of an AAV1 capsid and an AAV5 capsid. In embodiments of the embodiments described above, the rAAV particles comprise an AAV5 capsid. In embodiments of the embodiments described above, the rAAV particles comprise an AAV1 capsid. In embodiments of the above embodiments described above, the rAAV particles comprise at least one AAV1 ITR, AAV2 ITR, AAV3 ITR, AAV4 ITR, AAV5 ITR, AAV6 ITR, AAV7 ITR, AAV8 ITR, AAVrh8 ITR, AAV9 ITR, AAV10 ITR, AAVrh10 ITR, AAV11 ITR, AAV12 ITR, AAV-13 ITR, AAV-14 ITR, AAV-15 ITR, AAV-16 ITR, AAV.rh20 ITR, AAV.rh39 ITR, AAV.rh74 ITR, AAV.rhM4-1 ITR, AAV.hu37 ITR, AAV.Anc80 ITR, AAV DJ ITR, goat AAV ITR, bovine AAV ITR, or mouse AAV ITR. In some embodiments, the rAAV particles comprise ITRs from one AAV serotype and AAV capsid from another serotype. For example, the rAAV particles may comprise a therapeutic transgene flanked by at least one AAV2 ITR encapsidated into an AAV9 capsid. Such combinations may be referred to as pseudotyped rAAV particles.

### Recombinant Adeno-Associated Virus Particles

The methods described herein may be used to determine the presence of empty and/or partial capsids, and/or measure the relative amount empty and/or partial capsids in compositions of viral particles (e.g., recombinant adeno-associated virus (rAAV) particles).

In some embodiments, the rAAV genome is about 2500 bases to about 5500 bases in length. In some embodiments, the rAAV genome is between any of about 2500 bases and about 5500 bases, about 3000 bases and about 5500 bases, about 3500 bases and about 5500 bases, about 4000 bases and about 5500 bases, about 4500 bases and about 5500 bases, about 5000 bases and about 5500 bases, about 2500 bases and about 5000 bases, about 3000 bases and about 5000 bases, about 3500 bases and about 5000 bases, about 4000 bases and about 5000 bases, about 4500 bases and about 500 bases, about 2500 bases and about 4500 bases, about 3000 bases and about 4500 bases, about 3500 bases and about 4500 bases, about 4000 bases and about 4500, about 2500 bases and about 4000 bases, about 3000 bases and about 4000 bases, about 3500 bases and about 4000 bases, about 2500 bases and about 3500 bases, about 3000 bases and about 3500 bases, or about 2500 bases and about 3000 bases.

In some embodiments, the viral particle is a recombinant AAV particle comprising a nucleic acid comprising a transgene flanked by one or two ITRs (the rAAV genome). The nucleic acid is encapsidated in the AAV particle. The AAV particle also comprises capsid proteins. In some embodiments, the nucleic acid comprises the protein coding sequence(s) of interest (e.g., a therapeutic transgene) operatively linked components in the direction of transcription, control sequences including transcription initiation and termination sequences, thereby forming an expression cassette. The expression cassette is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810. Use of any AAV serotype is considered within the scope of the present invention. In some embodiments, a rAAV vector is a vector derived from an AAV serotype, including without limitation, AAV1, AAV2, AAV3, AAV4, AAV5, AAV6, AAV7, AAV8, AAV9, AAVrh.8, AAVrh.10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, a tyrosine capsid mutant, a heparin binding capsid mutant, an AAV2R471A capsid, an AAVAAV2/2-7m8 capsid, an AAV DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid, or the like. In some embodiments, the nucleic acid in the AAV comprises an ITR of AAV1, AAV2, AAV3, AAV4, AAV5, AAV6, AAV7, AAV8, AAV9, AAVrh.8, AAVrh10, AAV11, AAV12, AAV-13, AAV-14, AAV-15, AAV-16, AAV.rh20, AAV.rh39, AAV.rh74, AAV.rhM4-1, AAV.hu37, AAV.Anc80, AAV DJ, or the like. In some embodiments, the nucleic acid in the AAV comprises a goat AAV ITR, a bovine AAV ITR, or a mouse AAV ITR. In further embodiments, the rAAV particle comprises capsid proteins of AAV1, AAV2, AAV3, AAV4, AAV5, AAV6, AAV7, AAV8, AAV9, AAVrh.8, AAVrh.10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC1 1, AAV.HSC12, AAV.HSC13, AAV.HSC14, AAV.HSC15, AAV.HSC16, AAV2R471A, AAV2/2-7m8, AAV DJ, AAV2 N587A, AAV2 E548A, AAV2 N708A, AAV V708K or the like. In some embodiments, the rAAV particle comprises capsid proteins of goat AAV, AAV1/AAV2 chimera, bovine AAV, mouse AAV, or rAAV2/HBoV1 (chimeric AAV/human bocavirus virus 1). In further embodiments, the rAAV particle comprises capsid proteins of an AAV serotype from Clades A-F (Gao, et al. J. Virol. 2004, 78(12):6381). In some embodiments, the rAAV particle comprise capsid proteins selected from the group consisting of AAV1 capsid proteins and AAV5 capsid proteins. In some embodiments, the rAAV particle comprises capsid proteins of AAV5. In some embodiments, the rAAV particle comprises capsid proteins of AAV1. In some embodiments, the rAAV particle comprise capsid selected from the group consisting of an AAV1 capsid and an AAV5 capsid. In some embodiments, the rAAV particle comprises an AAV5 capsid. In some embodiments, the rAAV particle comprises an AAV1 capsid

Different AAV serotypes are used to optimize transduction of particular target cells or to target specific cell types within a particular target tissue (e.g., a diseased tissue). A rAAV particle can comprise viral proteins and viral nucleic acids of the same serotype or a mixed serotype. For example, a rAAV particle can comprise AAV9 capsid proteins and at least one AAV2 ITR or it can comprise AAV2 capsid proteins and at least one AAV9 ITR. In yet another example, a rAAV particle can comprise capsid proteins from both AAV9 and AAV2, and further comprise at least one AAV2 ITR. Any combination of AAV serotypes for production of a rAAV particle is provided herein as if each combination had been expressly stated herein.

In some embodiments, the rAAV particle comprises at least one AAV1 ITR and capsid protein from any of AAV2, AAV3, AAV4, AAV5, AAV6, AAV7, AAV8, AAV9, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAV2 ITR and capsid protein from any of AAV1, AAV3, AAV4, AAV5, AAV6, AAV7, AAV8, AAV9, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16. In some embodiments, the rAAV particle comprises at least one AAV3 ITR and capsid protein from any of AAV1, AAV2, AAV4, AAV5, AAV6, AAV7, AAV8, AAV9, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAV4 ITR and capsid protein from any of AAV1, AAV2, AAV3, AAV5, AAV6, AAV7, AAV8, AAV9, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAV5 ITR and capsid protein from any of AAV1, AAV2, AAV3, AAV4, AAV6, AAV7, AAV8, AAV9, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16. In some embodiments, the rAAV particle comprises at least one AAV6 ITR and capsid protein from any of AAV1, AAV2, AAV3, AAV4, AAV5, AAV7, AAV8, AAV9, AAVrh.8, AAVrh0, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and/or AAV.HSC16. In some embodiments, the rAAV particle comprises at least one AAV7 ITR and capsid protein from any of AAV1, AAV2, AAV3, AAV4, AAV5, AAV6, AAV8, AAV9, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAV8 ITR and capsid protein from any of AAV1, AAV2, AAV3, AAV4, AAV5, AAV6, AAV7, AAV9, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAV9 ITR and capsid protein from any of AAV1, AAV2, AAV3, AAV4, AAV5, AAV6, AAV7, AAV8, AAVrh.8, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAVrh8 ITR and capsid protein from any of AAV1, AAV2, AAV3, AAV4, AAV5, AAV6, AAV8, AAV9, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAVrh10 ITR and capsid protein from any of AAV1, AAV2, AAV3, AAV4, AAV5, AAV6, AAV7, AAV8, AAV9, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAV11 ITR and capsid protein from any of AAV1, AAV2, AAV3, AAV4, AAV5, AAV6, AAV7, AAV8, AAVrh8, AAV9, AAVrh10, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16. In some embodiments, the rAAV particle comprises at least one AAV12 ITR and capsid protein from any of AAV1, AAV2, AAV3, AAV4, AAV5, AAV6, AAV7, AAV8, AAV rh8, AAV9, AAVrh10, AAV11, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, and/or AAV.HSC16.

In some embodiments, the rAAV particle comprises an AAV5 capsid protein and an ITR selected from the group consisting of AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, an AAV12 ITR, an AAV-13 ITR, an AAV-14 ITR, an AAV-15 ITR, an AAV-16 ITR, an AAV.rh20 ITR, an AAV.rh39 ITR, an AAV.rh74 ITR, an AAV.rhM4-1 ITR, an AAV.hu37 ITR, an AAV.Anc80 ITR, an AAV DJ ITR, a goat AAV ITR, a bovine AAV ITR, and/or a mouse AAV ITR

Self-Complementary AAV Viral Genomes

In some aspects, the methods of the invention may be used to determine the presence of empty and/or partial capsids, and/or measure the relative amount empty and/or partial capsids in compositions of viral particles comprising a recombinant self-complementing genome. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a transgene). In some embodiments, the invention provides an AAV viral particle comprising an AAV genome, wherein the rAAV genome comprises a first heterologous polynucleotide sequence (e.g., a therapeutic transgene coding strand) and a second heterologous polynucleotide sequence (e.g., the noncoding or antisense strand of the therapeutic transgene) wherein the first heterologous polynucleotide sequence can form intrastrand base pairs with the second polynucleotide sequence along most or all of its length. In some embodiments, the first heterologous polynucleotide sequence and a second heterologous polynucleotide sequence are linked by a sequence that facilitates intrastrand basepairing; e.g., a hairpin DNA structure. Hairpin structures are known in the art, for example in siRNA molecules. In some embodiments, the first heterologous polynucleotide sequence and a second heterologous polynucleotide sequence are linked by a mutated ITR (e.g., the right ITR). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

Production of rAAV Vectors

Numerous methods are known in the art for production of rAAV vectors, including transfection, stable cell line production, and infectious hybrid virus production systems which include adenovirus-AAV hybrids, herpesvirus-AAV hybrids (Conway, JE el al., (1997) J. Virology 71(11):8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpesvirus, baculovirus, or a plasmid construct providing helper functions; 3) AAV rep and cap genes and gene products; 4) a transgene (such as a therapeutic transgene) flanked by at least one AAV ITR sequences; and 5) suitable media and media components to support rAAV production. In some embodiments, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. In some embodiments, the AAV helper functions are provided by adenovirus or HSV. In some embodiments, the AAV helper functions are provided by baculovirus and the host cell is an insect cell (e.g., Spodoptera frugiperda (Sf9) cells). In some embodiments, the p5 promoter of the nucleic acid encoding AAV rep and cap regions is located 3′ to the rep and/or cap coding region. In some embodiments, the nucleic acid encoding AAV rep and cap coding regions is plasmid pHLP, pHLP19, or pHLP09 (see U.S. Pat. Nos. 5,622,856; 6,001,650; 6,027,931; 6,365,403; 6,376,237; and 7,037,713; the content of each is incorporated herein in its entirety). In some embodiments, the AAV helper virus functions comprise adenovirus EIA function, adenovirus E1B function, adenovirus E2A function, adenovirus VA function and adenovirus E4 orf6 function.

Suitable rAAV production culture media of the present invention may be supplemented with serum or serum-derived recombinant proteins at a level of 0.5%-20/o (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

In some aspects, the invention provides methods for monitoring the removal of empty capsids during the purification of a composition of rAAV particles, the method comprising collecting a sample of the composition before and following one or more steps of the purification process and analyzing each collected sample for the relative amount of empty capsids according to the methods as described herein, wherein a decrease in the relative amount of empty capsids between the samples subsequently collected indicates removal of empty capsids from the preparation of rAAV particles.

In some aspects, the invention provides methods of monitoring the removal of empty capsids during the purification of a composition of rAAV particles, the method comprising: a) determining the relative amount of empty capsids according to any one of the methods described herein in a first sample collected before the purification process or before a step of the purification of the process, b) determining the relative amount of empty capsids according to a method as described herein in a second sample collected following the purification process or a step of the purification of the process, wherein a decrease in the relative amount of empty capsids between the second and first collected samples indicates removal of empty capsids from the preparation of rAAV particles.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

Purification of rAAV Vectors

At harvest, rAAV production cultures of the present invention may contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins. In addition, rAAV production cultures further include rAAV particles having an AAV capsid serotype selected from the group consisting of AAV1, AAV2, AAV3, AAV4, AAV5, AAV6, AAV7, AAV8, AAVrh8, AAV9, AAV10, AAVrh10, AAV11, AAV12, AAV13, AAV14, AAV15, AAV16, AAVrh20, AAV.rh39, AAV.Rh74, AAV.RHM4-1, AAV.hu37, AAV.Anc80, AAV.Anc80L65, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC1O, AAV.HSC11, AAV.HSC12, AAV.HSC13, AAV.HSC14, AAV.HSC15, or AAV.HSC16. In some embodiments, the rAAV production cultures further comprise empty AAV capsids (e.g., a rAAV particle comprising capsid proteins but no rAAV genome). In some embodiments, the rAAV production cultures further comprise rAAV particles comprising variant rAAV genomes (e.g., a rAAV particle comprising a rAAV genome that differs from an intact full-length rAAV genome). In some embodiments, the rAAV production cultures further comprise rAAV particles comprising truncated rAAV genomes. In some embodiments, the rAAV production cultures further comprise rAAV particles comprising AAV-encapsidated DNA impurities.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade AIHC Millipore Millistak+HC Pod Filter, and a 0.2 m Filter Opticap XL10 Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232; U.S. Pat. Nos. 6,989,264 and 8,137,948 and WO 2010/148143.

### Kits

The methods of this invention can be provided in the form of a kit. In some embodiments, provided herein is a kit for measuring the relative amount empty capsids in a composition of rAAV particles according to any one of the methods described herein. In some embodiments, the kit comprises chromatography columns and/or buffers for use in the method. In some embodiments, the kit comprises three or more reference standards wherein the three or more reference standards comprise known ratios of full capsids to empty capsids ranging from 1:0 to 0:1. In some embodiments, the three or more reference standards comprise known ratios of full capsids to empty capsids of any one of 1:0, 0.9:0.1, 0.8:0.2, 0.7:0.3, 0.6:0:4, 0.5:0.5, 0.4:0.6, 0.3:0.7, 0.2:0.8, 0.1:0.9, and 0:1. In some embodiments, the three or more reference standards include preparations with low levels of empty capsids (e.g., >75% full capsids), approximately equal amounts of full and empty capsids (e.g., ˜50% full capsids), and high levels of empty capsids (e.g., <25% full capsids).

### EXEMPLARY EMBODIMENTS

The invention provides the following nonlimiting embodiments.

1. A method to determine the presence of empty and/or partial capsids in a composition comprising recombinant adeno-associated virus (rAAV) particles, the method comprising:
a) subjecting the composition to chromatography under conditions to separate the rAAV particles from impurities in the composition in an eluate of the chromatography;
b) measuring the UV absorbance at about 250 nm to about 270 nm (A₂₅₀₋₂₇₀) and about 220 nm to about 240 nM (A₂₂₀₋₂₄₀) of the eluate,
c) plotting chromatograms of the A₂₅₀₋₂₇₀ and A₂₂₀₋₂₄₀ of the eluate,
d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the presence of empty and/or partial capsids in the composition.

2. The method of embodiment 1, wherein the UV absorbance at about 250 nm to about 270 nm is a UV absorbance at about 260 nm.

3. The method of embodiment 1 or 2, wherein the UV absorbance at about 220 nm to about 240 nm is a UV absorbance at about 230 nm.

4. The method of any one of embodiments 1-3, wherein a PA260/PA230 ratio less than the PA260/PA230 ratio of a pure sample of full rAAV capsids is indicative of empty and/or partial capsids and in the composition.

5. The method of any one of embodiments 1-3, wherein a PA260/PA230 ratio equal to or less than a PA260/PA230 ratio of a rAAV sample with a known percentage of empty and/or partial capsids is indicative of empty and/or partial capsids in the composition.

6. A method of measuring the relative amount of empty and/or partial capsids in a composition comprising rAAV particles, the method comprising:
a) subjecting the composition to chromatography under conditions to separate the rAAV particles from impurities in the composition in an eluate of the chromatography;
b) measuring the UV absorbance at about 250 nm to about 270 nm (A₂₅₀₋₂₇₀) and about 220 nm to about 240 nM (A₂₂₀₋₂₄₀) of the eluate,
c) plotting chromatograms of the A₂₅₀₋₂₇₀ and A₂₂₀₋₂₄₀ of the eluate,
d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the relative amount of empty capsids in the composition.

7. The method of embodiment 6, wherein the UV absorbance at about 250 nm to about 270 nm is a UV absorbance at about 260 nm.

8. The method of embodiment 6 or 7, wherein the UV absorbance at about 220 nm to about 240 nm is a UV absorbance at about 230 nm.

9. The method of any one of embodiments 6-8, wherein the relative amount of empty capsids in the composition is determined by comparing the PA260/PA230 ratio of the eluate with a linear relationship between the PA260/PA230 ratio and the percent full capsids established with a plurality of rAAV preparations with different known percentages of full capsids.

10. The method of embodiment 9, wherein the linear relationship between the PA260/PA230 ratio and the percent full capsids is determined by plotting the PA260/PA230 ratio versus the percent of full capsids of the plurality of rAAV preparations.

11. The method of embodiment 9 or 10, wherein the linear relationship is represented by the equation % full capsids=(PA260/PA230−a)/h, wherein a is the PA260/PA230 of empty capsids and b is the slope of the linear equation determined from the plot of the PA260/PA230 ratio versus the percent of full capsids for the plurality of rAAV preparations.

12. The method of any one of embodiments 9-11, wherein the plurality of rAAV preparations with different known percentages of full capsids comprises three or more rAAV preparations wherein the three or more rAAV preparations comprise known ratios of full capsids to empty capsids ranging from 1:0 to 0:1.

13. The method of any one of embodiments 9-12, wherein the plurality of rAAV preparations comprise rAAV capsids of the same serotype as the rAAV particles in the composition.

14. The method of any one of embodiments 9-13, wherein the full capsids of the plurality of rAAV preparations comprise viral genomes that are at least about 90% in size compared to viral genomes of the rAAV particles in the composition.

15. The method of any one of embodiments 9-14, wherein the full capsids of the plurality of rAAV preparations comprise viral genomes that are the same as the viral genomes of the rAAV particles in the composition.

16. The method of any one of embodiments 9-15, wherein the plurality of rAAV preparations comprise capsids of the same serotype and the full capsids comprise the same viral genomes as the rAAV particles in the composition.

17. The method of any one of embodiments 1-16, wherein the chromatography is size exclusion chromatography, ion exchange chromatography, affinity chromatography, mixed mode chromatography, hydrophobic interaction chromatography, or apatite chromatography.

18. The method of any one of embodiments 1-17, wherein the chromatography utilizes a column chromatography.

19. The method of any one of embodiments 1-18, wherein the chromatography is high performance liquid chromatography (HPLC) or ultra-high performance liquid chromatography (UHPLC).

20. The method of any one of embodiments 1-19, wherein the chromatography is size exclusion chromatography.

21. A method of measuring the relative amount of empty and/or partial capsids in a composition comprising rAAV particles, the method comprising:
a) subjecting the composition to size exclusion chromatography under conditions to separate the rAAV particles from impurities in the composition in an eluate of the chromatography;
b) measuring the UV absorbance at about 250 nm to about 270 nm (A₂₅₀₋₂₇₀) and about 220 nm to about 240 nM (A₂₂₀₋₂₄₀) of the eluate,
c) plotting chromatograms of the A₂₅₀₋₂₇₀ and A₂₂₀₋₂₄₀ of the eluate,
d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the relative amount of empty capsids in the composition.

22. The method of embodiment 21, wherein the UV absorbance at about 250 nm to about 270 nm is a UV absorbance at about 260 nm.

23. The method of embodiment 21 or 22, wherein the UV absorbance at about 220 nm to about 240 nm is a UV absorbance at about 230 nm.

24. The method of any one of embodiments 21-23, wherein the relative amount of empty capsids is determined by comparing the PA260/PA230 ratio of the purified rAAV sample with a linear relationship between the PA260/PA230 ratio and the percent full capsids established with a plurality of rAAV preparations with different known percentages of full capsids.

25. The method of any one of embodiments 21-24, wherein the linear relationship between the PA260/PA230 ratio and the percent full capsids is determined by plotting the PA260/PA230 ratio versus the percent of full capsids of the plurality of rAAV preparations.

26. The method of any one of embodiments 21-25, wherein the linear relationship is represented by the equation % full capsids=(PA260/PA230−a)/h, wherein a is the PA260/PA230 of empty capsids and b is the slope of the linear equation determined from the plot of the PA260/PA230 ratio versus the percent of full capsids for the plurality of rAAV preparations.

27. The method of any one of embodiments 24-26, wherein the plurality of rAAV preparations with different known percentages of full capsids comprises three or more rAAV preparations wherein the three or more rAAV preparations comprise known ratios of full capsids to empty capsids ranging from 1:0 to 0:1

28. The method of any one of embodiments 24-27, wherein the plurality of rAAV preparations comprise rAAV capsids of the same serotype as the rAAV particles in the composition.

29. The method of any one of embodiments 24-28, wherein the full capsids of the plurality of rAAV preparations comprise viral genomes that are at least about 90% in size compared to viral genomes of the rAAV particles in the composition.

30. The method of any one of embodiments 24-29, wherein the full capsids of the plurality of rAAV preparations comprise viral genomes that are the same as the viral genomes of the rAAV particles in the composition.

31. The method of any one of embodiments 24-30, wherein the plurality of rAAV preparations comprise capsids of the same serotype and the full capsids comprise the same viral genomes as the rAAV particles in the composition.

32. The method of any one of embodiments 20-31, wherein the size exclusion chromatography utilizes a size exclusion chromatography column.

33. The method of embodiment 32, wherein the size exclusion chromatography column comprises particles about 3 μm to about 10 μm in diameter.

34. The method of embodiment 32 or 33, wherein the size exclusion chromatography column comprises particles about 5 μm in diameter.

35. The method of any one of embodiments 32-34, wherein the size exclusion chromatography column comprises particles with pores of about 100 Å to about 1000 Å in size.

36. The method of any one of embodiments 32-35, wherein the size exclusion chromatography column comprises particles with pores of about 500 Å in size.

37. The method of any one of embodiments 33-36, wherein the particles comprise silica.

38. The method of any one of embodiments 32-37, wherein the column has an internal diameter of between about 4.0 to about 7.8 mM and a length of between about 50 mm to about 300 mm, and in particular about 7.8 mm by about 300 mm, about 4.6 mm by about 100 mm, or about 4.6 mm by about 150 mm.

39. The method of any one of embodiments 32-38, wherein the size exclusion chromatography further utilizes a guard column.

40. The method of embodiment 39, wherein the guard column comprises the same particles as the size exclusion chromatography column.

41. The method of embodiment 39 or 40, wherein the guard column has an internal diameter of between about 4.0 to about 7.8 mM and a length of between about 30 mm to about 50 mm, and in particular is about 7.8 mm by about 50 mm, or about 4.6 by about 50 mm.

42. The method of any one of embodiments 20-41, wherein a mobile phase of the size exclusion chromatography comprises phosphate buffered saline (PBS).

43. The method of embodiment 42, wherein the PBS comprises
about 100 mM to about 200 mM NaCl,
about 1 mM to about 5 mM KCl,
about 5 mM to about 20 mM Na₂HPO₄, and
about 1 mM to about 3 mM KH₂PO₄.

44. The method of embodiment 42 or 43, wherein the PBS comprises about 137 mM NaCl,
about 2.7 mM KCl,
about 10 mM Na₂HPO₄, and
about 1.8 mM KH₂PO₄.

45. The method of any one of embodiments 42-44, wherein the PBS has a pH of about 6.5 to about 7.5 or about 7.0.

46. The method of embodiment 20-45, wherein the chromatography is performed at a flow rate of about 0.5 mL/minute to about 1.0 mL/minute, about 0.7 mL/minute to about 0.8 mL/minute, or about 0.75 mL/minute.

47. The method of any one of embodiments 1-46 wherein the chromatography is performed at about 4° C. to about 35° C.

48. The method of any one of embodiments 1-47 wherein the chromatography is performed at about 25° C., about 30° C., or about 35° C.

49. The method of any one of embodiments 1-48, wherein about 5 μL to about 500 μL or about 10 μL to about 75 μL are subjected to the chromatography.

50. The method of any one of embodiments 1-49, wherein the titer of rAAV in the composition is about 1×10¹¹ to about 1×10⁴ capsid particles/mL (cp/mL) or about 5×10¹² cp/mL.

51. The method of any one of embodiments 1-50, wherein the titer of rAAV in the composition is about 1×10¹⁰ to about 1×10¹⁴ viral genomes/mL (vg/mL).

52. The method of any one of embodiments 1-51, wherein greater than about 70% of the rAAV particles in the composition are full rAAV capsids.

53. The method of any one of embodiments 1-52, wherein greater than about 70% to about 95% of the rAAV particles in the composition are full rAAV capsids.

54. The method of any one of embodiments 1-53, wherein the rAAV particles in the composition have been purified using one or more purification steps.

55. The method of any one of embodiments 1-54, wherein the recombinant viral particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAV13 capsid, an AAV14 capsid, an AAV15 capsid, an AAV16 capsid, an AAVrh20 capsid, an AAV.rh39 capsid, an AAV.Rh74 capsid, an AAV.RHM4-1 capsid, an AAV.hu37 capsid, an AAV.Anc80 capsid, an AAV.Anc80L65 capsid, an AAV.PHP.B capsid, an AAV2.5 capsid, an AAV2tYF capsid, an AAV3B capsid, an AAV.LK03 capsid, an AAV.HSC1 capsid, an AAV.HSC2 capsid, an AAV.HSC3 capsid, an AAV.HSC4 capsid, an AAV.HSC5 capsid, an AAV.HSC6 capsid, an AAV.HSC7 capsid, an AAV.HSC8 capsid, an AAV.HSC9 capsid, an AAV.HSC10 capsid, an AAV.HSC11 capsid, an AAV.HSC12 capsid, an AAV.HSC13 capsid, an AAV.HSC14 capsid, an AAV.HSC15 capsid, an AAV.HSC16 capsid, an AAV2R471A capsid, an AAV2/2-7m8 capsid, an AAV DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid rAAV2/HBoV1 (chimeric AAV/human bocavirus virus 1).

56. The method of any one of embodiments 1-55, wherein the recombinant viral particle comprises an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, an AAV12 ITR, an AAV-13 ITR, an AAV-14 ITR, an AAV-15 ITR, an AAV-16 ITR, an AAV.rh20 ITR, an AAV.rh39 ITR, an AAV.rh74 ITR, an AAV.rhM4-1 ITR, an AAV.hu37 ITR, an AAV.Anc80 ITR, an AAV DJ ITR, a goat AAV ITR, a bovine AAV ITR, or a mouse AAV ITR.

57. The method of any one of embodiments 1-56, wherein the rAAV genome is 2500 bases to 5500 bases in length.

58. The method of any one of embodiments 1-57, wherein the recombinant viral particles comprise a self-complementary AAV (scAAV) genome.

59. The method of any one of embodiments 6-58, wherein the rAAV particles is selected from the group consisting of rAAV5 particles and rAAV1 particles.

60 The method of any one of embodiments 6-59, wherein the rAAV particles are rAAV5 particles

61. The method of embodiment 60, wherein the relative amount of empty capsids is calculated from the equation PA260/PA230=0.0057 (relative percent full capsids)+0.1137.

62 The method of any one of embodiments 6-59, wherein the rAAV particles are rAAV1 particles

63. The method of embodiment 62, wherein the relative amount of empty capsids is calculated from the equation PA260/PA230=0.0054 (relative percent full capsids)+0.0886.

64. A method of monitoring the removal of empty capsids during the purification of a composition of rAAV particles, the method comprising collecting a sample of the composition before and following one or more steps of the purification process and analyzing each collected sample for the relative amount of empty capsids according to the method of any one of embodiments 1-63, wherein a decrease in the relative amount of empty capsids between the samples subsequently collected indicates removal of empty capsids from the preparation of rAAV particles.

65. A method of monitoring the removal of empty capsids during the purification of a composition of rAAV particles, the method comprising:
a) determining the relative amount of empty capsids according to the method of any one of embodiments 1-63 in a first sample collected before the purification process or before a step of the purification of the process,
b) determining the relative amount of empty capsids according to the method of any one of embodiments 1-37 in a second sample collected following the purification process or a step of the purification of the process,
wherein a decrease in the relative amount of empty capsids between the second and first collected samples indicates removal of empty capsids from the preparation of rAAV particles.

66. A kit for measuring the relative amount empty capsids in a composition of rAAV particles according the methods of any one of embodiments 1-65.

67. The kit of embodiment 66, wherein the kit comprises chromatography columns and/or buffers for use in the methods of any one of embodiments 1-65.

68. The kit of embodiment 66 or 67, wherein the kit comprises three or more reference standards wherein the three or more reference standards comprise known ratios of full capsids to empty capsids ranging from 1:0 to 0:1.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Initial Size-Exclusion Chromatography Column Screening

The following example describes an initial screen to identify a column suitable for performing size-exclusion chromatography (SEC) on AAV particles. The selection of a suitable column to perform SEC analysis of AAV capsids is primarily dictated by the molecular properties of the capsids. The molecular weight of AAV capsids is 3.5-6.0 megadaltons and the capsids have a diameter of 20-25 nm. Therefore, SEC resins with a pore size from 300 to 1,000 Å (30 to 100 nm) were anticipated to be suitable for separating monomeric AAVs from aggregated species as well as from low molecular weight species and small molecules that are expected to be present in a sample. To avoid any possible sieving effect, an analytical column with a large inner diameter packed with a stationary phase consisting of a relatively large particle size would be preferred. Several vendors including Waters, Sepax and Wyatt manufacture columns that may be suitable for this purpose. Sepax SRT® SEC columns are packed with spherical high purity silica coated with nanometer thick hydrophilic and neutral films and these materials have shown promise for separation of virus and virus-like particles.

### Results

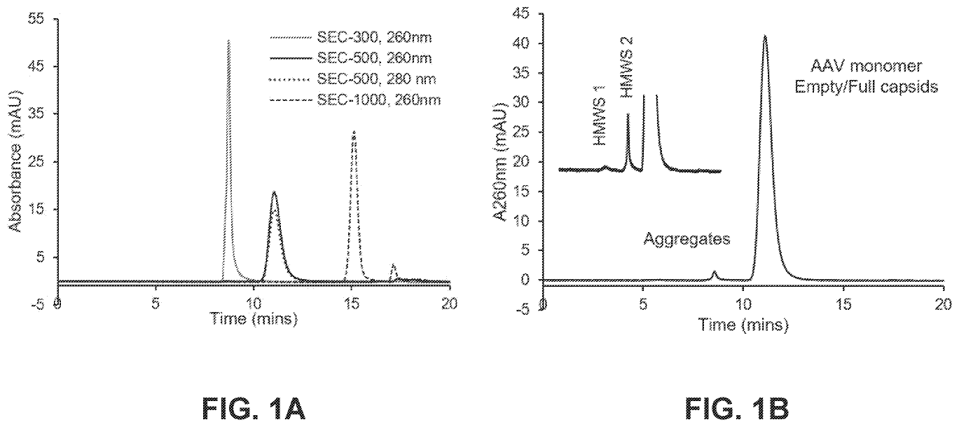

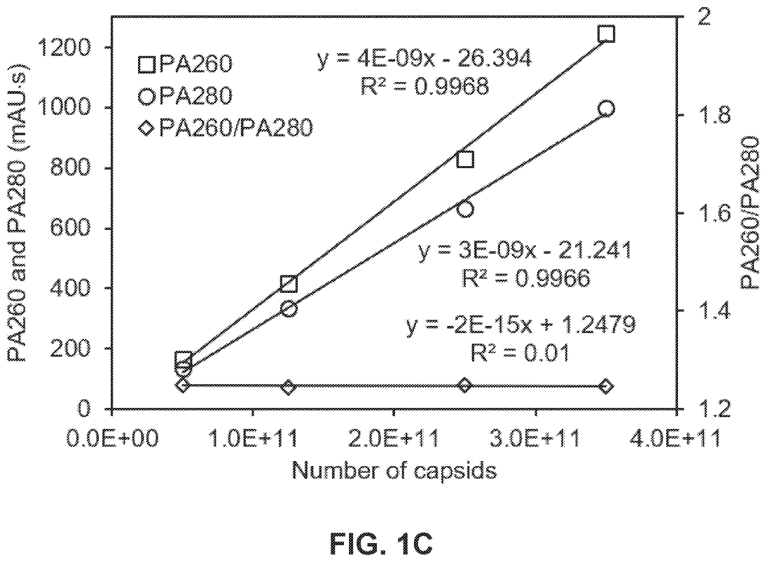

Three columns with preferred column dimensions (300×7.8 mm) and particle size (5 μm), but different pore sizes (300, 500 and 1,000 Å) were evaluated for their performance to analyze AAV5 containing samples using phosphate buffered saline (PBS) as the mobile phase. The chromatograms of AAV5 samples are shown in FIGS. 1A-1C.

The monomeric vectors eluted from the column with the retention time increasing in the order of increased pore size, manifesting the diffusion characteristics of the solute inside and outside of different micropores (FIG. 1A). The monomer peak observed in the elution profile obtained for the SEC-300 column is sharp but slightly skewed, and the trace amount of aggregates, which is expected to elute prior to the monomer peak, was not observed. The elution profile obtained for the SEC-1000 column (FIG. 1A) shows the monomer eluting too close to the peak representing low molecular weight species (buffer peaks). The SRT® SEC-500 column with a 500 Å pore size was therefore selected for further development as the retention time, peak shape and resolution are satisfactory. As shown in FIG. 1B, the peak area representing the monomer is higher when the signal is monitored at 260 nm compared to 280 nm resulting in a peak area ratio (PA260/PA280) of approximately 1.25 which is indicative of a sample containing mostly full capsids. The minor peaks eluting at approximately 5.8 mins and 8.6 mins are AAV aggregates (0.5% for HMWS 1 and 1.7% for HMWS 2), indicating that the method is capable of detecting low level aggregates. When different volumes (10-75 μL of 5×10¹² cp/mL) of the DS sample were injected, excellent linearity was observed (R²=0.997) for the peak areas at 260 nm and 280 nm in relation to the number of capsids injected (FIG. 1C), suggesting that the titer of the sample could be derived. The PA260/PA280 ratio remained unchanged over the range tested.

### Example 2: Size-Exclusion Chromatography with Dual-Wavelength Detection for the AAV Spiked Samples

The following example describes the use of size-exclusion chromatography with dual-wavelength detection (SEC-DW) to analyze preparations of AAV capsids.

### Materials and Methods

Preparation of empty capsids, full capsids and spiked samples. Empty capsids were purified using a strong anion exchange HQ column. The full capsids were isolated by AVB Sepharose® affinity column and HQ anion exchange column, and further purified by cesium chloride density gradient centrifugation to remove any remaining empty and partial capsids and achieve a high purity.

Approximately 20 mL of purified AAV5 virus (˜5×10¹³-1×10¹⁴ total vector genomes) was diluted to 30 mL with 20 mM NaH₂PO₄, 400 mM NaCl, 4 mM MgCl₂, pH 7.5, to which about 17.8 g of cesium chloride (CSCl₂) was added to bring the final density to 1.35 g/mL. The solution was centrifuged in a Beckman centrifuge equipped with a 70ti rotor at 5,000 rpm overnight at 15° C. Two bands were visible with a dual high intensity fiber light lamp. The upper band and lower band were collected via syringes as the empty and full capsids, respectively. The samples were dialyzed against 1×PBS using 10K MWCO Slide-a-Lyzers™ (Thermo Scientific, MA) and then against the DS formulation buffer.

The empty and full capsids were characterized by analytical ultracentrifugation (AUC) and droplet digital PCR (ddPCR) and qPCR (Bio-Rad, Hercules, CA) to determine the capsid distribution and vector genome concentration. The concentration of the empty capsid preparation was determined by AAV titration ELISA (Progen, Biotechnik) or A₂₈₀. The results are summarized in Table 1.

TABLE 1
Capsid concentration, vector genome concentration and
subpopulation distribution of empty and full capsids

| | | Vector | | | |
|---|---|---|---|---|---|
| | Capsids | genome | Empty | Partial | Full |
| | conc. | conc. | capsids | capsids | capsids |
| Samples | (cp/mL) | (vg/mL) | (%) | (%) | (%) |
| Empty capsids | 1.21 × 10¹³ | 2.50 × 10¹¹ | 98 | 2 | 0 |
| Full capsids | 4.95 × 10¹² | 4.50 × 10¹² | 0 | 9 | 91 |

A series of high performance liquid chromatography (HPLC) standards and spiked samples containing various amounts of full capsid were prepared by mixing the empty and full capsids at defined ratios based on their capsid concentrations (cp/mL). These materials were used to develop the SEC-DW method for empty and full capsids. In addition, for comparison purposes, the samples were evaluated using orthogonal approaches such as UV measurements, AUC characterization and cryogenic electron microscopy (Cryo-EM) analyses (see Example 3).

Extinction coefficients and UV measurements. The extinction coefficient of empty capsids at 280 nm (ε_{280,capsid}) was calculated based on the absorptivity of the aromatic amino acids present in the primary sequences of VP1, VP2 and VP3, using a ratio of VP1:VP2:VP3 of 5:5:50 per capsid. The extinction coefficient of empty capsids at 260 nm (ε_{260,capsid}) was obtained by converting the 280, capsid using a previously published conversion factor of 0.59 (Sommer, J. M. et al., Mol Ther 2003, 7 (1), 122-8). The molecular weight (MW) of empty capsids was calculated based on the sequence information and the aforementioned protein ratio. The extinction coefficient of the transgene at 260 nm (ε_{260, gene}) and its MW were calculated based on the gene sequence, and the extinction coefficient at 280 nm (ε_{280,gene}) was then derived with a conversion factor of 0.555 (Sommer, J. M. et al., Mol Ther 2003, 7 (1), 122-8). The extinction coefficients at 260 nm and 280 nm of full capsids were the sum of the extinction coefficients of capsid and transgene at each wavelength (e.g., ε_{260, full capsid}=ε_{260, capsid}+ε_{260, gene}). The extinction coefficients of empty and full capsids are summarized in Table 2. The predicted A₂₆₀/A₂₈₀ ratio of the different samples used in the study was calculated based on the relative amounts of empty and full capsids present in the samples.

TABLE 2
Theoretical extinction coefficients
of empty capsids and full capsids

| ε_{280, empty capsid} | ε_{260, empty capsid} | ε_{280, full capsid} | ε_{260, full capsid} |
|---|---|---|---|
| (M⁻¹ · cm⁻¹) | (M⁻¹ · cm⁻¹) | (M⁻¹ · cm⁻¹) | (M⁻¹ · cm⁻¹) |
| 6,768,550.0 | 3,993,444.5 | 21,731,437.4 | 30,953,601.9 |

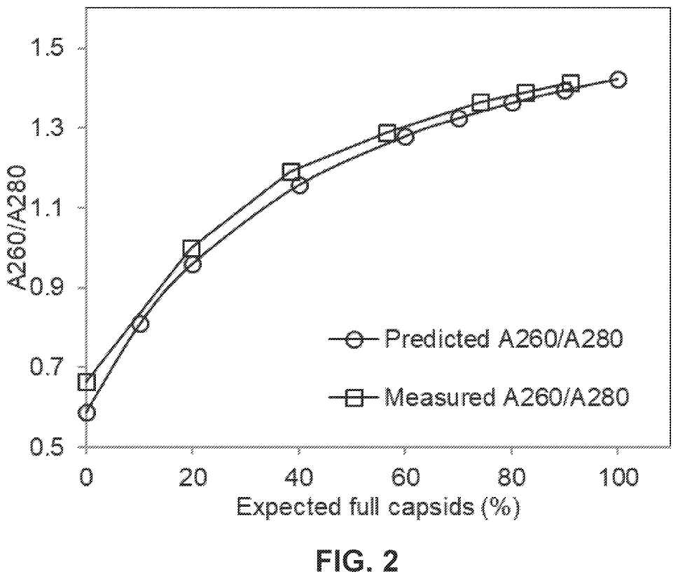

UV absorbance of empty, full and spiked samples was measured using a nanodrop spectrometer (Thermo Fisher, MA). A₂₆₀/A₂₈₀ data was plotted against the expected percent full capsids and compared with the predicted A₂₆₀/A₂₈₀ data (see FIG. 2).

SEC-dual-wavelength. AAV capsids (generally 25 μL or 50 μL per injection) were loaded onto a size exclusion column (Sepax SRT® SEC-500, 5 μm, 50 Å, 7.8×300 mm, Delaware) equipped with a guard column (SRT® SEC-500, 5 μm, 50 Å, 7.8×50 mm) equilibrated at 25° C. with Dulbecco's phosphate buffered saline without CaCl₂, without MgCl₂ (DPBS). In addition to the SEC-500 column two additional columns (Sepax SRT® SEC-300, 5 μm, 30 Å, 7.8×300 mm equipped with a guard column SRT® SEC-300, 5 μm, 30 Å, 7.8×50 mm and Sepax SRT® SEC-1000, 5 μm, 100 Å, 7.8×300 mm) equipped with a guard column SRT® SEC-1000, 5 μm, 100 Å, 7.8×50 mm) were evaluated for comparison. All columns were run under the same conditions.

The temperature of the autosampler was set at 5° C. The capsids were eluted with DPBS, pH 7.0 at a flow rate of 0.75 mL/min using an Agilent 1200 series HPLC (Agilent, CA). The eluate was monitored at 260 nm, 280 nm and 230 nm, and data was acquired with the Agilent OpenLab software.

The peak areas of monomeric AAV at 230 nm, 260 nm and 280 nm were used for calculating the peak area (PA) ratios (e.g., PA260/PA230 and PA260/PA280). The correlation of the peak area ratios with the percent full capsids was established using the HPLC standards and the spiked samples containing various amounts of full capsids.

### Results

SEC with Dual-Wavelength Detection for the AAV Spiked Samples

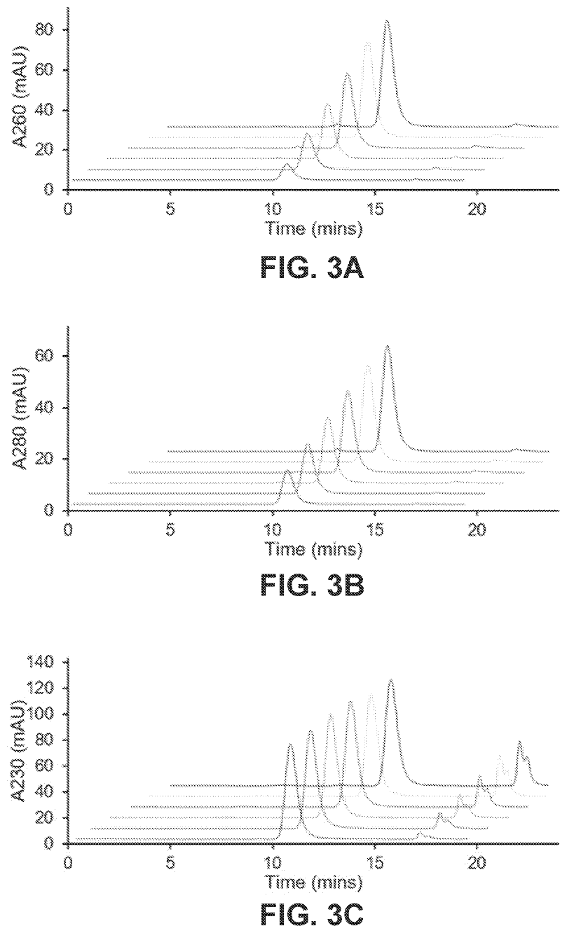

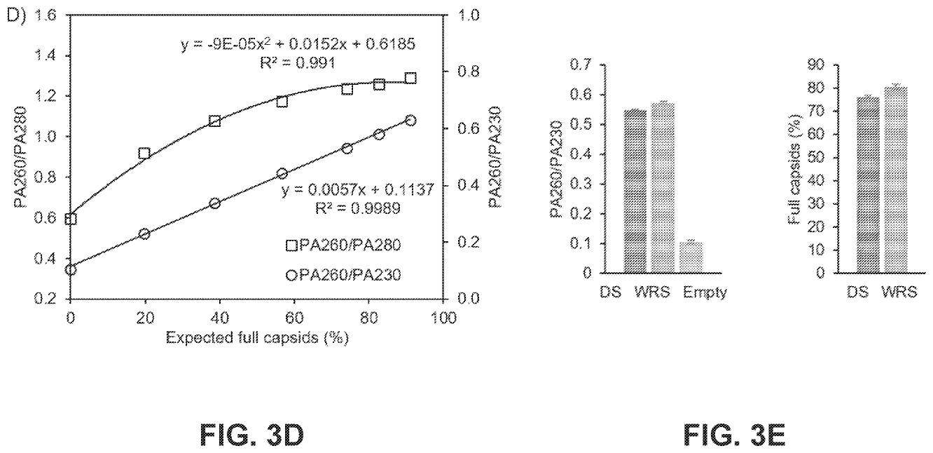

As described in Example 1, the various capsid standards and samples were initially monitored at 260 nm and 280 nm, where encapsidated nucleic acids and capsid proteins have their absorbance maxima, respectively (see FIG. 1A) (Porterfield, J. Z. and Zlotnick, A., Virology 2010, 407 (2), 281-8). The SEC chromatograms of the HPLC standard samples in FIG. 3A and FIG. 3B show that the monomers were well-resolved from the small aggregate peaks and the PA260/PA280 ratios obtained for the standard samples were plotted against the expected value for the percent full capsids calculated based on the ratio of the empty and full capsid stock solutions combined to prepare the samples (FIG. 3D). As shown in FIG. 3D, the PA260/PA280 ratio approached an asymptote when the full capsids accounted for more than 70% of total capsids. As a result, the method had limited capability to determine the percentage full capsids with appropriate accuracy and precision when the full capsid content is greater than 70%. A similar limitation was previously observed when using UV spectroscopy to monitor the A₂₆₀/A₂₈₀ ratios (Sommer, J. M. et al., Mol Ther 2003, 7 (1), 122-8) and the A₂₆₀/A₂₈₀ ratios of the spiked samples are shown in FIG. 2. As gene therapy candidates often have more than 70% full capsids in the drug substance and drug product, owing to the continuous improvements of the upstream and downstream processes, a method capable of accurately monitoring percent full capsids greater than 90% is required.

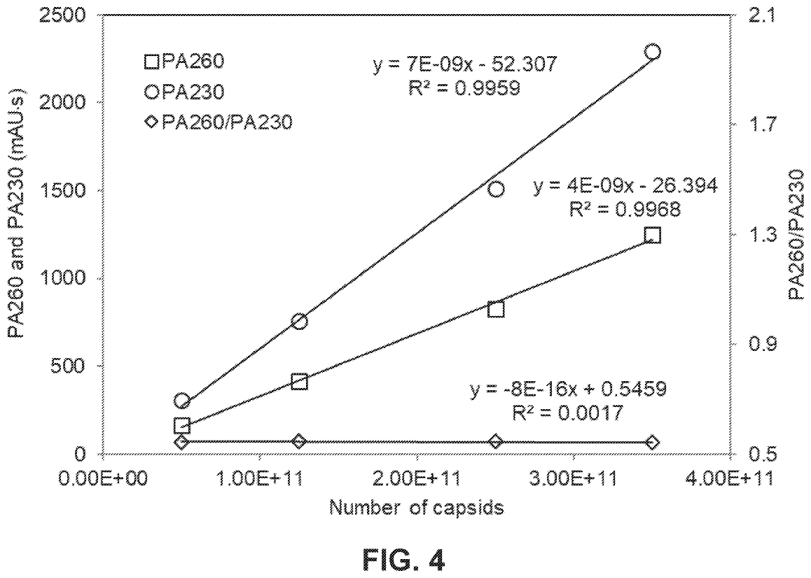

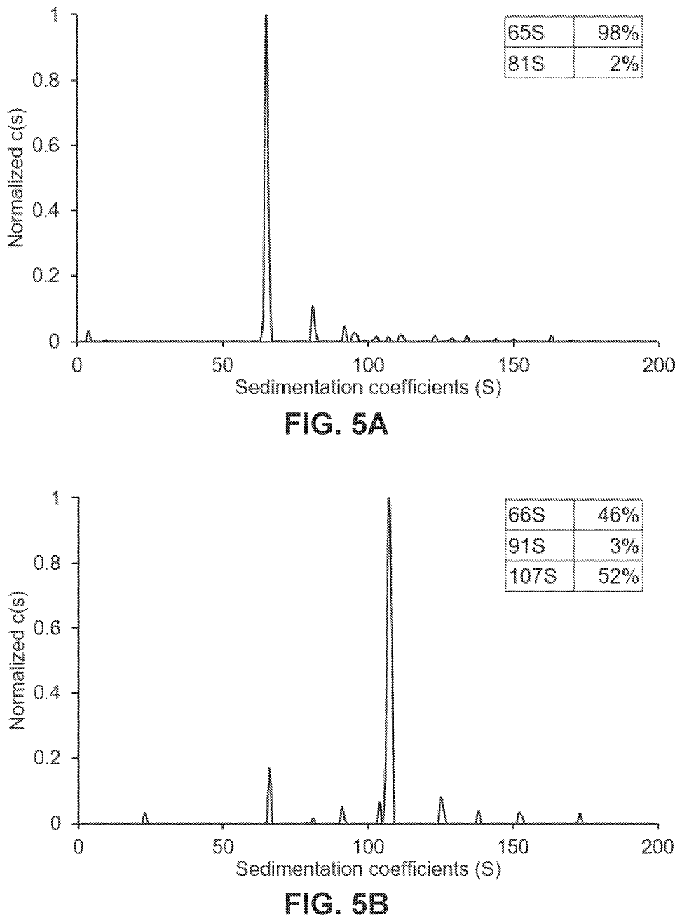

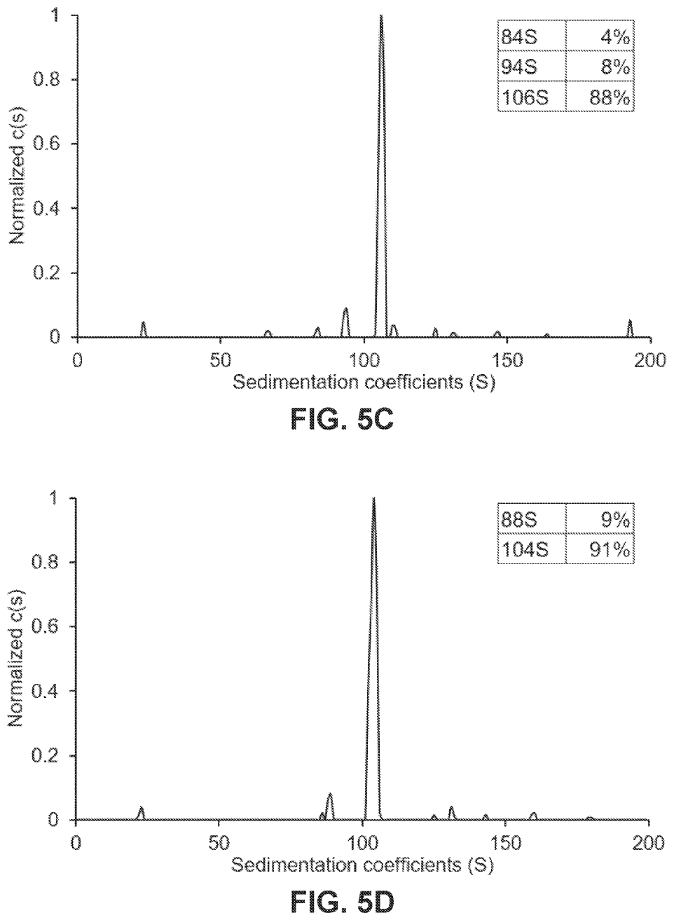

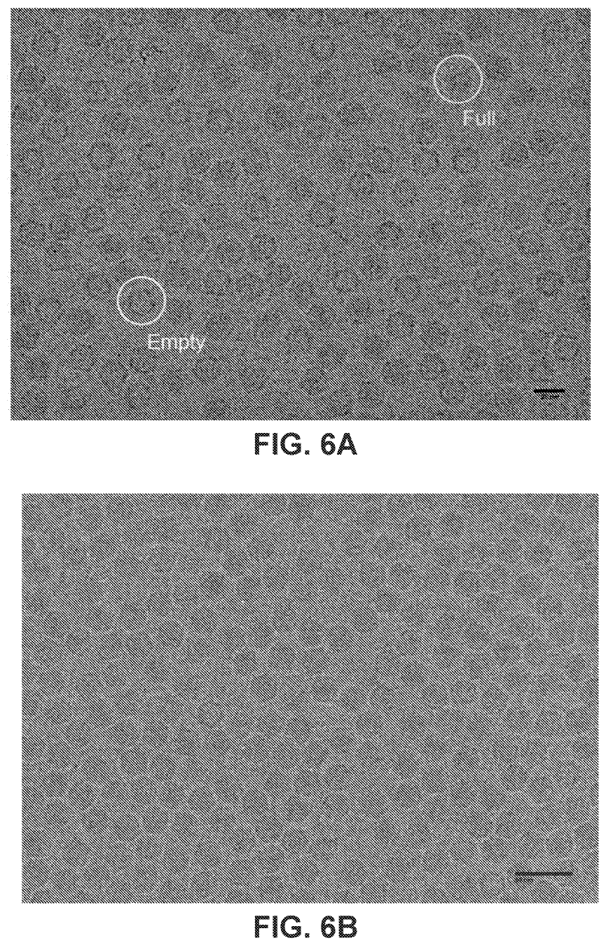

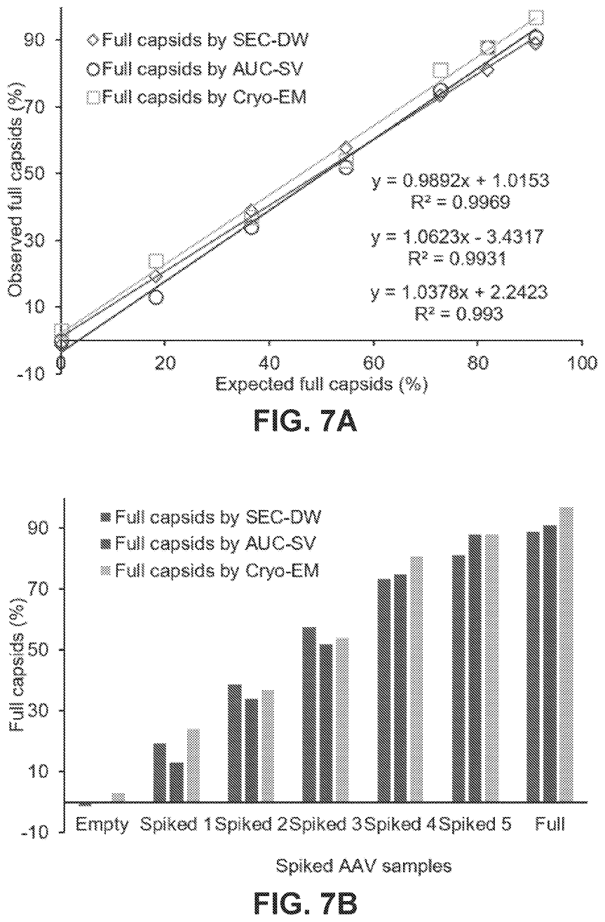

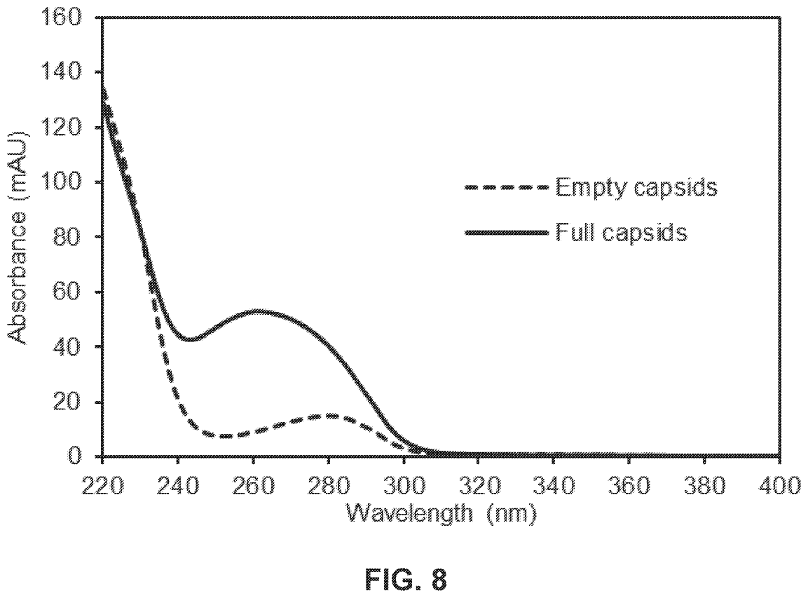

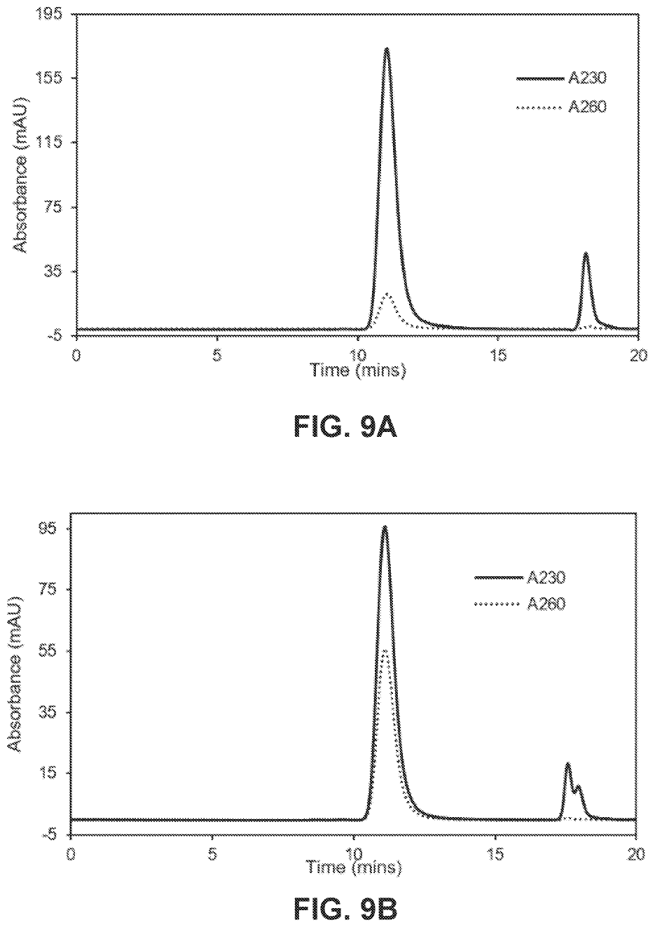

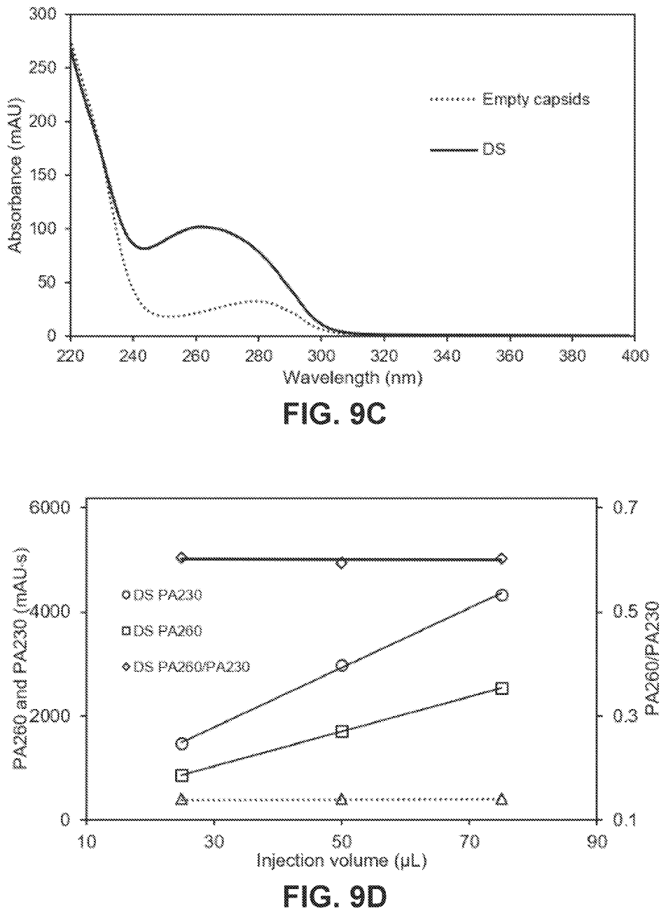

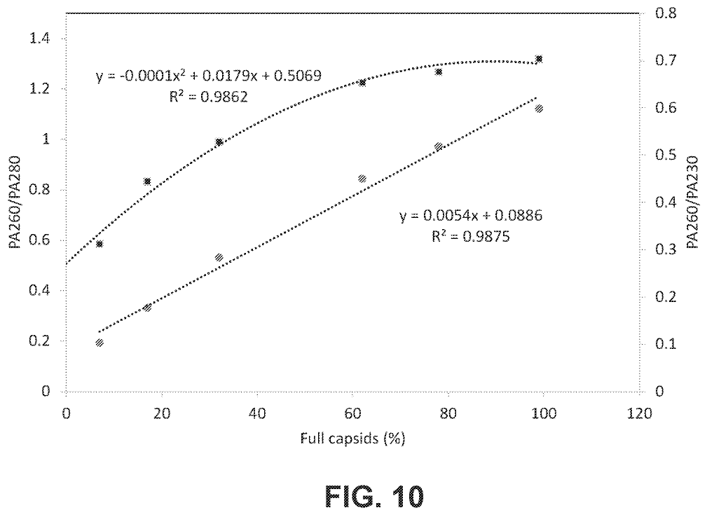

To extend the range of the method the use of other wavelengths for protein detection was evaluated. Without wishing to be bound by theory, it was hypothesized that the absorbance of capsid proteins would be much more intense and becomes dominant at the 230 nm wavelength, while the contributory absorbance of transgenes would be limited. FIG. 3C shows the SEC traces of the standards at 230 nm. As hypothesized, the peak area monitored at 260 nm and 230 nm was proportional to the column loading and the PA260/PA230 ratio remains constant over the range tested, confirming that the intrinsic PA260/PA230 ratio was independent of the capsid concentration (see FIG. 4). As shown in FIG. 4, the PA260/PA230 ratio was unchanged across the loading range tested. By using this new dual wavelength for detection and calculation of the peak area ratios, a linear relationship between PA260/PA230 and percent full capsids was established (y=0.0057x+0.1137). The goodness of fit was excellent, judged by the R² of 0.999 (FIG. 3D).

The ability to use a linear curve fit when using the PA260/PA230 ratio versus the percent full capsid is critical as such a change allows the extension of the assay range beyond 70% full capsids. The full capsid sample used in this study contained 91% full and 9% partial capsids, and therefore it was not possible to evaluate the capability of the method above this level. However, the assay range may potentially be extend to 100% full capsids, given the linear relationship observed. Due to the nature of SEC chromatography this analysis can be performed using only a small volume (e.g., 10-50 μL) at capsid concentrations of ˜5×10¹² cp/mL. In addition, the method also normalizes the pH and ionic strength of the sample to match the pH and ionic strength of the mobile phase, and therefore minimizes their impact on the absorbance of transgenes (Wilfinger, W.W. et al., Biotechniques 1997, 22 (3), 474-6, 478-81).

### Specificity, Reproducibility, and Robustness of SEC-DW

As described above, the SEC-DW method separated the AAV capsids from the excipients and/or buffer components. Thus, the components in the formulation buffer are not expected to have any impact on the PA260/PA230 ratio. The Bio-Rad's gel filtration standard containing thyroglobulin, γ-globulin, ovalbumin, myoglobin, and vitamin B₁₂ was used to estimate what size of proteins may interfere with the analysis. Bovine thyroglobulin eluted at the retention time windows of 8-13 minutes, indicating that only proteins of this size (MW 670,000 Da) and higher would potentially interfere with the quantitation of empty and full capsids. This type of protein impurities is not expected to be present at a level that is impactful in the samples post affinity and/or strong anion exchange column purification. The injections of the mobile phase blank after 14 injections of AAV samples showed no detectable peaks by both wavelengths near the retention time of monomeric AAV5, indicating no carryover from the previous injections.

The SEC-DW method was used to determine the percentage of full capsids in a composition representative of the drug substance (DS) and in a working reference standard (WRS). Samples were compared against a standard curve generated using HPLC standards containing different ratios of the empty and full capsids (FIG. 3D). The analysis was then performed over multiple assay occasions (different mobile phase lots) on different days. The PA260/PA230 values were 0.55 (n=8), 0.57 (n=4), and 0.11 (n=6) for the DS, WRS and empty capsid samples. The results indicated that the DS and the WRS samples contained approximately 76% and 83% full capsids, respectively (FIG. 3E), similar to the results obtained by AUC-SV. The percent full capsids for the empty capsid sample was 0% (the actual value calculated based on the linear equation established was −1.1%). The % RSD values of the PA260/PA230 ratios and % full capsids for the DS sample (n=8) and the WRS sample (n=4) were less than 1%, demonstrating that the results were highly reproducible over different assays, different mobile phase lots, and different days.

The spiked samples prepared separately using the empty and full capsids stocks led to a similar linear equation (y=0.0057x+0.1195) between the PA260/PA230 ratios and the percent full capsids and a R² value of 0.999. The same slope and similar intercept again manifest the limited variations of the SEC-DW method.

To gain understanding of the effect of changes in method parameters, the pH and flow rate of the mobile phase were deliberately varied during the development (three pH values at 6.6, 7.0, and 7.5, and three flow rates at 0.70, 0.75, and 0.80 mL/min). The PA260/PA230 values of the same DS sample were 0.55 under all three pH conditions and were 0.54 at the three flow rates. The percent full capsids were in the range of 74.8-76.4%, again using the linear equation established. These results demonstrated that the SEC-DW method was robust within the variations tested.

### Example 3: Comparison of Determination of Percent of Full Capsids by SEC-DW, AUC-SV and Cryo-EM

The following example describes experiments comparing the ability of size-exclusion chromatography with dual-wavelength detection (SEC-DW), sedimentation velocity analytical ultracentrifugation (AUC-SV), and cryogenic electron microscopy (Cryo-EM) to determine the percentage of full capsids in AAV preparations.

### Materials and Methods

Sedimentation Velocity Analytical Ultracentrifugation (AUC-SV). Empty capsids, spiked samples, and full capsids were buffer exchanged into 1×PBS, pH 7.2 (Invitrogen) using 10K MWCO Slide-a-Lyzers™ or Amicon 10K MWCO centrifugal filters (Millipore Sigma, MO). The absorbance at 260 nm (A₂₆₀) of these samples was measured using a nanodrop spectrometer (Thermo Scientific, MA), to ensure the samples were sufficiently concentrated for the sedimentation experiments (0.2≤A₂₆₀≤0.6).

For the sedimentation velocity experiment, the AUC sample (˜400 μL) was loaded into the sample sector of a two sector 1.2 cm Charcoal-filled Epon centerpiece (Beckman Coulter) and PBS (˜410 μL) was loaded into the reference sector. The cells were inserted in a four-hole rotor and equilibrated at full vacuum and 20° C. for at least one hour in the centrifuge (Optima™ XL-I, Beckman Coulter). The vectors were sedimented at 20,000 rpm and the absorbance of the vectors was scanned at 260 nm in a continuous mode.

AUC-SV data analysis. The absorbance data was loaded into SEDFIT and fitted with a continuous c(s) distribution model. The meniscus was floated, and the friction ratio was fixed at 1.0 while fitting the data to the Lamm equation, with time-invariant (TI) and radius-invariant (RI) noise correction. The second-derivative regularization was applied to the fitting with a confidence level of 0.68. A range of 1-200 for sedimentation coefficients was used with a resolution of 200. The published density and viscosity values of PBS were used. The relative abundance of each species in unit of detection was converted to relative abundance in molar concentration by correcting the absorbance according to Beer's Law. The content of each species was reported as a percent of the total. The details of the data analysis have been described in Burnham, B. et al. (Burnham, B. et al., Hum Gene Ther Methods 2015, 26 (6), 228-42).

Cryogenic electron microscopy (Cryo-EM) Grid preparation. C-flat holey carbon grids were cleaned with 20 mA for 30 seconds in a Pelco EasiGlow plasma cleaner. Vitrified specimens were prepared by loading a grid into a manual plunger (EMS-002 Rapid Immersion Freezer), adding 4 μL of virus to the grid, immediately one-side blotting the grid for 2 seconds, and freezing the sample in liquid ethane.

Cryo-EM Imaging. Electron microscopy was performed using a Titan Krios® electron microscope operated with an accelerating voltage at 300 kV and equipped with a K3 direct electron detector. Data was collected using SerialEM at 105,000× nominal magnification (pixel size of 0.83 Å) at 2.5 μm defocus and a dose of ˜48.0 e⁻/Å².

Cryo-EM data analysis. An in-house data analysis software program developed at the Cryo-EM Core Facility of UMass Medical School was used to recognize and count the empty and full capsids. For quantitation of the percent full capsids, many images were processed and a total of approximately 2,200-8,700 vectors were analyzed and counted as empty and full capsids for each spiked sample. The content of full capsids from all the images analyzed for each sample was reported as a percent of the total capsids.

### Results

### Percent Full Capsids by AUC-SV

To further examine the suitability of the SEC-DW method for empty and full capsids, the spiked samples were analyzed by AUC-SV using a previously developed method (see Burnham, B. et al., Hum Gene Ther Methods 2015, 26 (6), 228-42). The spiked samples were first buffer exchanged to 1×PBS, transferred into AUC cells, and sedimented at 20,000 rpm. The sedimentation of vectors was monitored at 260 nm along the centrifugal field in a continuous mode, and the acquired data were fitted to the Lamm equation using the computer program SEDFIT (Brown, P. H. et al., Biophys. J 2006, 90 (12), 4651-61). The representative sedimentation coefficient distribution c(s) plots for four samples (empty, two spiked samples and full capsids) are shown in Error! Reference source not found.

Different species including empty, partial and full capsids, as well as high molecular weight species were detected and identified based on their sedimentation coefficients (approximately 64-66S for empty capsids, 80-90S for partial capsids, and 101-107S for full capsids with a transgene of ˜4,370 nucleotides). In addition, trace amount of high molecular weight species were evident. These high molecular weight species were not included in the quantitation of the overall empty, partial and full capsids. Integration of each peak resulted in the relative percentage of each species in unit of detection, which was converted to the molar concentration of each species according to Beer's Law due to the considerable differences in the extinction coefficients at 260 nm of the individual species (Burnham, B. et al., Hum Gene Ther Methods 2015, 26 (6), 228-42). FIG. 5A shows the sedimentation coefficient distribution plot for the subpopulations of the empty capsid sample (98% empty capsids (65S) and 2% partial capsids (81S)). The results for the full capsid sample purified via cesium chloride density gradient centrifugation are shown in FIG. 5D and indicated that the sample contained 91% full capsids (104S) and 9% partial capsids (88S). The partial capsids with a sedimentation coefficient of 88S represented capsids harboring fragmented genomes of approximately 2,700 nucleotides (Nass, S. A. et al., Mol Ther Methods Clin Dev 2018, 9, 33-46). On a separate AUC-SV analysis, the subpopulations of the empty sample were 99% empty (65S) and 1% partial capsids (83S), the subpopulations of the full capsid sample were 8% partial (86S) and 92% full capsids (101S), demonstrating the consistency between the AUC-SV runs.

FIG. 5B and FIG. 5C show samples which were prepared by mixing the full and empty capsid containing samples. The % full capsid for these samples was calculated as 55% and 82% full capsids, respectively, based on the assigned values for the full and empty capsid samples and the combined volumes. The full capsid contents were 52% and 88% by AUC-SV for these two samples. The results from the SEC-DW method were in very good agreement with the AUC-SV results (approximately 58% and 81% for these two samples). A small fraction of partial capsids was present for all samples, particularly in the full capsid sample despite the stringent purification efforts. The presence of partial capsids manifests the challenge of producing drug substance that mainly consist of full capsids. The partial capsids cannot be discerned by anion exchange chromatography, SEC and transmission electron microscopy methods, but are well distinguished by AUC-SV. In addition, the samples recovered from AUC-SV were re-analyzed by SEC-DW directly with a 25 μL injection. Similar chromatographic profiles and percent full capsid results were obtained, confirming that all the capsids remained intact post AUC-SV experiments.

### Percent Full Capsids by Cryo-EM

Cryogenic electron microscopy (Cryo-EM) is an electron microscopy technique that can measure empty and full capsids in their frozen hydrated state (Subramanian, S. et al., Hum Gene Ther 2019, 30 (12), 1449-1460). Vitrified capsids were prepared by freezing them in liquid ethane, and electron microscopy images were collected using a Titan Krios. One of the Cryo-EM images taken for the spiked sample containing approximately 55% full capsids is shown in FIG. 6A, in which the representative full capsid (displaying significant internal density) and empty capsid (absence of internal density) are circled and both the empty and full capsids are similar in size (approximately 24 nm). FIG. 6B was selected from the images for the full capsid sample, and almost no empty capsids can be identified. For each spiked sample, at least 2,200 particles from multiple images were counted using in-house data analysis software (Cryo-EM core facility of UMASS Medical School). The results from all images analyzed were combined to calculate the percentage of full capsids and ensure statistical significance. Partial capsids were treated as full capsids by Cryo-EM analyses in this study, although they can be identified by Cryo-EM via 3D classification (Subramanian, S. et al., Hum Gene Ther 2019, 30 (12), 1449-1460). The percentages of full capsids for the empty, spiked samples and full capsid sample are 3, 24, 37, 54, 81, 88 and 97%. The number of images analyzed, the total number of full capsids, the total number of empty capsids, the number of particles counted with low confidence (artifact, background signal, etc.), and the percent full capsids in the samples by Cryo-EM are summarized in Table 3. The SEC-DW results were similar to the percent full capsids measured by Cryo-EM, and thus SEC-DW provides a viable option for good manufacturing practice testing.

TABLE 3
Percent full capsids determined by Cryo-EM for the spiked samples

| | | | | | Total | |
|---|---|---|---|---|---|---|
| | Number | Number | | | number of | |
| | of | of | Particles | Number | full and | Full |
| | full | empty | with low | of | empty | capsids |
| Samples | capsids | capsids | confidence | images | capsids | (%) |
| Empty | 85 | 3287 | 247 | 48 | 3372 | 2.5 |
| Spiked 1 | 1322 | 4210 | 447 | 114 | 5532 | 23.9 |
| Spiked 2 | 1482 | 2563 | 466 | 99 | 4045 | 36.6 |
| Spiked 3 | 2487 | 2120 | 258 | 45 | 4607 | 54.0 |
| Spiked 4 | 1805 | 426 | 533 | 104 | 2231 | 80.9 |
| Spiked 5 | 2633 | 345 | 220 | 64 | 2978 | 88.4 |
| Full | 8447 | 260 | 317 | 95 | 8707 | 97.0 |

### Comparison of Percent Full Capsids Determined by SEC-DW, AUC-SV and Cryo-IM

To scientifically validate the SEC-DW method, the percentage of full capsids determined by SEC-DW was compared with the results from two state-of-the-art high-resolution methods: AUC-SV and Cryo-EM which can accurately measure the percent full capsids. These three methods all characterize the AAV samples in their native state: in solution passing through the column for SEC-DW, free in solution in a centrifugal field for AUC-SV, and in frozen hydrated state after flash-freezing for Cryo-EM. The percent full capsids of the spiked samples determined by SEC-DW, AUC-SV and Cryo-EM are illustrated in FIGS. 7A-7B show the percentage of full capsids in spiked samples as determined by SEC-DW, AUC-SV and Cryo-EM. The results from all three methods demonstrated excellent linearity between the observed and expected percent full capsids (FIGS. 7A-7B show the percentage of full capsids in spiked samples as determined by SEC-DW, AUC-SV and Cryo-EM.), judged by the R² values from the linear regressions (0.997, 0.993, and 0.993 for SEC-DW, AUC-SV and Cryo-EM, respectively). The slopes of the linear regressions were 0.989, 1.062, and 1.038 for SEC-DW, AUC-SV and Cryo-EM, indicating that the SEC-DW method did not have any significant bias when compared to the AUC-SV and Cryo-EM methods. Partial capsids were treated as full capsids by Cryo-EM in this study, thus, the reported full capsids for each sample was slightly higher than those values from AUC-SV (FIGS. 7A-7B show the percentage of full capsids in spiked samples as determined by SEC-DW, AUC-SV and Cryo-EM.). Overall, the percent full capsids by SEC-DW based on PA260/PA230 agreed very well with the results from AUC-SV and Cryo-EM (FIGS. 7A-7B show the percentage of full capsids in spiked samples as determined by SEC-DW, AUC-SV and Cryo-EM.) across the whole range tested (0-91% full capsids, or 0-98% empty capsids), demonstrating the SEC-DW method was accurate and reliable.

### SEC-DW Results of a Second Composition (DS) and Corresponding Empty Capsids

To examine if the SEC-DW method can be generalized, an AAV5 DS sample with a different transgene but the same AAV serotype (AAV5) was profiled by SEC-DW together with the corresponding enriched empty capsids (FIGS. 9A-9D). The PA260/PA230 ratio of the enriched empty capsids was 0.12, which was about the same as the AAV5 empty capsids used for the method development (PA260/PA230=0.11), demonstrating the intrinsic nature of this ratio for the same serotype capsid. The PA260/PA230 of the second DS sample was 0.58, estimated to be approximately 82% full capsids using the linear relationship shown in FIG. 3D. Again, the PA260/PA230 ratios were the same across the loading range tested for both empty capsids and DS samples. Based on these results, it is envisioned that this SEC-DW method is generalizable to other AAV5 samples, and potentially to other serotypes. The relationship between PA260/PA230 and percent full capsids can be applied to the sample testing directly, with an appropriate assay control such as a WRS sample in place to ensure the assay performance.

### Conclusion

The SEC-DW method described herein for determining the relative amounts of empty and full capsid used HPLC equipment that is readily available in development and quality control labs, directly quantified the percent full capsids in a sample, required small sample amounts (10-75 μL of 5×10¹² cp/mL sample per injection), provided the highest throughput among the three methods, and can be qualified/validated for use under current good manufacturing practice. The SEC-DW method did not need additional sample preparations such as denaturation and labeling, and the impact of matrices were effectively eliminated during the size exclusion elution.

The presence of partial capsids is thought to be inevitable using the current manufacturing processes. The gene sequences packaged in the partial capsids do contribute to the absorbance at 260 nm and 230 nm, and therefore introduce some variations to the reportable results. Some of these variations are built in the linear equation initially established and therefore the impact of partial capsids on the quantitation of the percent full capsids is limited. The light scattered by a spherical solute is inversely proportional to λ (Russell, S. et al., Lancet 2017, 390 (10097), 849-860), according to Rayleigh approximation. The light scattering can be corrected for absorbance at 230 nm and 260 nm using the wavelength dependent extrapolation. The absorbance spectra of empty and full capsids at 220-400 nm were extracted from the data acquired by the photodiode array (PDA) detector and no significant light scattering was observed based on the minimal to neglectable absorbance at 320-360 nm (see FIG. 8, FIGS. 9A-9D).

The SEC-DW method described herein was established using various AAV5 DS samples and it is anticipated to be applicable for testing in-process samples as long as no interference from any other macromolecules that may elute as monomeric AAVs. The in-process samples can be readily concentrated with a concentrator to ensure a proper capsid concentration. This SEC-DW method can be applied similarly to other AAV serotypes once the linear relationship of PA260/PA230 vs. percent full capsids is established. Considering the low volume of sample and the instrument required, the SEC-DW method is anticipated to prove useful for determining empty and full capsid contents on a readily accessible instrument platform in a high throughput manner.

### Example 4: Size-Exclusion Chromatography with Dual-Wavelength Detection for the AAV1 Spiked Samples

The following example describes the use of size-exclusion chromatography with dual-wavelength detection (SEC-DW) to analyze preparations of AAV1 capsids with a total of vector genome of 3.468 kb.

### Materials and Methods

Preparation of empty capsids, full capsids and spiked samples. Empty capsids were procured from a vendor (Vigene Biosciences). The full capsids were purified internally. The full capsids were characterized by analytical ultracentrifugation (AUC) and droplet digital PCR (ddPCR) (Bio-Rad, Hercules, CA) to determine the capsid distribution and vector genome concentration. The concentration of the empty capsid preparation was determined by ELISA and the percent empty capsids was determined by TEM. The results are summarized in Table 4.

TABLE 4
Capsid concentration, vector genome concentration and
subpopulation distribution of empty and full capsids

| | | Vector | | | |
|---|---|---|---|---|---|
| | Capsids | genome | Empty | Partial | Full |
| | conc. | conc. | capsids | capsids | capsids |
| Samples | (cp/mL) | (vg/mL) | (%) | (%) | (%) |
| Empty capsids | 2.26 × 10¹² | NA | 92.6 | NA | NA |
| Full capsids | 1.4 × 10¹³ | 1.4 × 10¹³ | 0 | 1 | 99 |

A series of high-performance liquid chromatography (HPLC) standards and spiked samples containing various amounts of full capsid were prepared by mixing the empty and full capsids at defined ratios based on their capsid concentrations (cp/mL). These materials were used to develop the SEC-DW method for empty and full capsids.

SEC-dual-wavelength. AAV capsids (generally 40 μL or 50 μL per injection) were loaded onto a size exclusion column (Sepax SRT® SEC-500, 5 μm, 50 Å, 7.8×300 mm, Delaware) equipped with a guard column (SRT® SEC-500, 5 μm, 50 Å, 7.8×50 mm) equilibrated at 25° C. with Dulbecco's phosphate buffered saline without CaCl₂), without MgCl₂ (DPBS).

The temperature of the autosampler was set at 5° C. The capsids were eluted with DPBS, pH 7.2 at a flow rate of 0.75 mL/min using an Agilent 1200 series HPLC (Agilent, CA). The eluate was monitored at 260 nm, 280 nm and 230 nm, and data was acquired with the Agilent OpenLab software.

The peak areas of monomeric AAV at 230 nm, 280 nm and 260 nm were used for calculating the peak area (PA) ratios (e.g., PA260/PA230, and PA260/PA280). The correlation of the peak area ratios with the percent full capsids was established using the HPLC standards and the spiked samples containing various amounts of full capsids.

### Results

SEC with Dual-Wavelength Detection for the AAV1 Spiked Samples

The peak area monitored at 260 nm and 230 nm was proportional to the column loading and the PA260/PA230 ratio remains constant over the range tested, confirming that the intrinsic PA260/PA230 ratio was independent of the capsid concentration. By using this new dual wavelength for detection and calculation of the peak area ratios, a linear relationship between PA260/PA230 and percent full capsids was established (y=0.0054x+0.0886). The goodness of fit was excellent, judged by the R² of 0.9875 (FIG. 10).

The ability to use a linear curve fit when using the PA260/PA230 ratio versus the percent full capsid is critical as such a change allows the extension of the assay range beyond 70% full capsids. It is worthy to note that the assay range would still be significantly extended compared to the use of A260 and A280 for detection, as no plateau for PA260/PA230 was observed when the amount of full capsids is greater than 70%, while a plateau was evident for PA260/PA280 when the amount of full capsids is greater than 70%.

What is claimed is:

1. A method to determine the presence of empty and/or partial capsids in a composition comprising recombinant adeno-associated virus (rAAV) particles, the method comprising:
a) subjecting the composition to chromatography under conditions to separate the rAAV particles from impurities in the composition in an eluate of the chromatography;
b) measuring the UV absorbance at about 250 nm to about 270 nm (A₂₅₀₋₂₇₀) and about 220 nm to about 240 nM (A₂₂₀₋₂₄₀) of the eluate,
c) plotting chromatograms of the A₂₅₀₋₂₇₀ and A₂₂₀₋₂₄₀ of the eluate,
d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the presence of empty and/or partial capsids in the composition.

2. The method of claim 1, wherein the UV absorbance at about 250 nm to about 270 nm is a UV absorbance at about 260 nm.

3. The method of claim 1 or 2, wherein the UV absorbance at about 220 nm to about 240 nm is a UV absorbance at about 230 nm.

4. The method of any one of claims 1-3, wherein a PA260/PA230 ratio less than the PA260/PA230 ratio of a pure sample of full rAAV capsids is indicative of empty and/or partial capsids and in the composition.

5. The method of any one of claims 1-3, wherein a PA260/PA230 ratio equal to or less than a PA260/PA230 ratio of a rAAV sample with a known percentage of empty and/or partial capsids is indicative of empty and/or partial capsids in the composition.

6. A method of measuring the relative amount of empty and/or partial capsids in a composition comprising rAAV particles, the method comprising:
a) subjecting the composition to chromatography under conditions to separate the rAAV particles from impurities in the composition in an eluate of the chromatography;
b) measuring the UV absorbance at about 250 nm to about 270 nm (A₂₅₀₋₂₇₀) and about 220 nm to about 240 nM (A₂₂₀₋₂₄₀) of the eluate,
c) plotting chromatograms of the A₂₅₀₋₂₇₀ and A₂₂₀₋₂₄₀ of the eluate,
d) integrating the area under the peaks representing the rAAV particles for the A₂₅₀₋₂₇₀ and A₂₂₀₋₂₄₀ plots, wherein the peak area ratio at A₂₅₀₋₂₇₀ and A₂₂₀₋₂₄₀ (PA260/PA230) is indicative of the relative amount of empty capsids in the composition.

7. The method of claim 6, wherein the UV absorbance at about 250 nm to about 270 nm is a UV absorbance at about 260 nm.

8. The method of claim 6 or 7, wherein the UV absorbance at about 220 nm to about 240 nm is a UV absorbance at about 230 nm.

9. The method of any one of claims 6-8, wherein the relative amount of empty capsids in the composition is determined by comparing the PA260/PA230 ratio of the eluate with a linear relationship between the PA260/PA230 ratio and the percent full capsids established with a plurality of rAAV preparations with different known percentages of full capsids.

10. The method of claim 9, wherein the linear relationship between the PA260/PA230 ratio and the percent full capsids is determined by plotting the PA260/PA230 ratio versus the percent of full capsids of the plurality of rAAV preparations.

11. The method of claim 9 or 10, wherein the linear relationship is represented by the equation % full capsids=(PA260/PA230−a)/b, wherein a and b are constants determined from the plot the PA260/PA230 ratio versus the percent of full capsids for the plurality of rAAV preparations.

12. The method of any one of claims 9-11, wherein the plurality of rAAV preparations with different known percentages of full capsids comprises three or more rAA V preparations, wherein the three or more rAAV preparations comprise known ratios of full capsids to empty capsids ranging from 1:0 to 0:1.

13. The method of any one of claims 9-12, wherein the plurality of rAAV preparations comprise rAAV capsids of the same serotype as the rAAV particles in the composition.

14. The method of any one of claims 9-13, wherein the full capsids of the plurality of rAAV preparations comprise viral genomes that are at least about 90% in size compared to viral genomes of the rAAV particles in the composition.

15. The method of any one of claims 9-14, wherein the full capsids of the plurality of rAAV preparations comprise viral genomes that are the same as the viral genomes of the rAAV particles in the composition.

16. The method of any one of claims 9-15, wherein the plurality of rAAV preparations comprise capsids of the same serotype and the full capsids comprise the same viral genomes as the rAAV particles in the composition.

17. The method of any one of claims 1-16, wherein the chromatography is size exclusion chromatography, ion exchange chromatography, affinity chromatography, mixed mode chromatography, hydrophobic interaction chromatography, or apatite chromatography.

18. The method of any one of claims 1-17, wherein the chromatography utilizes a column chromatography.

19. The method of any one of claims 1-18, wherein the chromatography is high performance liquid chromatography (HPLC) or ultra-high performance liquid chromatography (UHPLC).

20. The method of any one of claims 1-19, wherein the chromatography is size exclusion chromatography.

21. A method of measuring the relative amount of empty and/or partial capsids in a composition comprising rAAV particles, the method comprising:
a) subjecting the composition to size exclusion chromatography under conditions to separate the rAAV particles from impurities in the composition in an eluate of the chromatography;
b) measuring the UV absorbance at about 250 nm to about 270 nm (A₂₅₀₋₂₇₀) and about 220 nm to about 240 nM (A₂₂₀₋₂₄₀) of the eluate,
c) plotting chromatograms of the A₂₅₀₋₂₇₀ and A₂₂₀₋₂₄₀ of the eluate,
d) integrating the area under the peaks representing the rAAV particles for the A₂₅₀₋₂₇₀ and A₂₂₀₋₂₄₀ plots, wherein the peak area ratio at A₂₂₀₋₂₇₀ and A₂₂₀₋₂₄₀ (PA260/PA230) is indicative of the relative amount of empty capsids in the composition.

22. The method of claim 21, wherein the UV absorbance at about 250 nm to about 270 nm is a UV absorbance at about 260 nm.

23. The method of claim 21 or 22, wherein the UV absorbance at about 220 nm to about 240 nm is a UV absorbance at about 230 nm.

24. The method of any one of claims 21-23, wherein the relative amount of empty capsids is determined by comparing the PA260/PA230 ratio of the purified rAAV sample with a linear relationship between the PA260/PA230 ratio and the percent full capsids established with a plurality of rAAV preparations with different known percentages of full capsids.

25. The method of any one of claims 21-24, wherein the linear relationship between the PA260/PA230 ratio and the percent full capsids is determined by plotting the PA260/PA230 ratio versus the percent of full capsids of the plurality of rAAV preparations.

26. The method of any one of claims 21-25, wherein the linear relationship is represented by the equation % full capsids=(PA260/PA230−a)/b, wherein a is the PA260/PA230 of empty capsids and b is the slope of the linear equation determined from the plot of the PA260/PA230 ratio versus the percent of full capsids for the plurality of rAAV preparations.

27. The method of any one of claims 24-26, wherein the plurality of rAAV preparations with different known percentages of full capsids comprises three or more rAAV preparations wherein the three or more rAAV preparations comprise known ratios of full capsids to empty capsids ranging from 1:0 to 0:1

28. The method of any one of claims 24-27, wherein the plurality of rAAV preparations comprise rAAV capsids of the same serotype as the rAAV particles in the composition.

29. The method of any one of claims 24-28, wherein the full capsids of the plurality of rAAV preparations comprise viral genomes that are at least about 90% in size compared to viral genomes of the rAAV particles in the composition.

30. The method of any one of claims 24-29, wherein the full capsids of the plurality of rAAV preparations comprise viral genomes that are the same as the viral genomes of the rAAV particles in the composition.

31. The method of any one of claims 24-30, wherein the plurality of rAAV preparations comprise capsids of the same serotype and the full capsids comprise the same viral genomes as the rAAV particles in the composition.

32. The method of any one of claims 20-31, wherein the size exclusion chromatography utilizes a size exclusion chromatography column.

33. The method of claim 32, wherein the size exclusion chromatography column comprises particles about 3 μm to about 10 μm in diameter.

34. The method of claim 32 or 33, wherein the size exclusion chromatography column comprises particles about 5 μm in diameter.

35. The method of any one of claims 32-34, wherein the size exclusion chromatography column comprises particles with pores of about 100 Å to about 1000 Å in size.

36. The method of any one of claims 32-35, wherein the size exclusion chromatography column comprises particles with pores of about 500 Å in size.

37. The method of any one of claims 33-36, wherein the particles comprise silica.

38. The method of any one of claims 32-37, wherein the column has an internal diameter of between about 4.0 to about 7.8 mM and a length of between about 50 mm to about 300 mm, and in particular about 7.8 mm by about 300 mm, about 4.6 mm by about 100 mm, or about 4.6 mm by about 150 mm.

39. The method of any one of claims 32-38, wherein the size exclusion chromatography further utilizes a guard column.

40. The method of claim 39, wherein the guard column comprises the same particles as the size exclusion chromatography column.

41. The method of claim 39 or 40, wherein the guard column has an internal diameter of between about 4.0 to about 7.8 mM and a length of between about 30 mm to about 50 mm, and in particular is about 7.8 mm by about 50 mm, or about 4.6 by about 50 mm.

42. The method of any one of claims 20-41, wherein a mobile phase of the size exclusion chromatography comprises phosphate buffered saline (PBS).

43. The method of claim 42, wherein the PBS comprises
about 100 mM to about 200 mM NaCl,
about 1 mM to about 5 mM KCl,
about 5 mM to about 20 mM Na₂HPO₄, and
about 1 mM to about 3 mM KH₂PO₄.

44. The method of claim 42 or 43, wherein the PBS comprises
about 137 mM NaCl,
about 2.7 mM KCl,
about 10 mM Na₂HPO₄, and
about 1.8 mM KH₂PO₄

45. The method of any one of claims 42-44, wherein the PBS has a pH of about 6.5 to about 7.5 or about 7.0.

46. The method of claim 20-45, wherein the chromatography is performed at a flow rate of about 0.5 mL/minute to about 1.0 mL/minute, about 0.7 mL/minute to about 0.8 mL/minute, or about 0.75 mL/minute.

47. The method of any one of claims 1-46 wherein the chromatography is performed at about 4° C. to about 35° C.

48. The method of any one of claims 1-47 wherein the chromatography is performed at about 25° C., about 30° C., or about 35° C.

49. The method of any one of claims 1-48, wherein about 5 μL to about 500 μL or about 10 μL to about 75 μL are subjected to the chromatography.

50. The method of any one of claims 1-49, wherein the titer of rAAV in the composition is about 1×10¹¹ to about 1×10⁴ capsid particles/mL (cp/mL) or about 5×10¹ cp/mL.

51. The method of any one of claims 1-50, wherein the titer of rAAV in the composition is about 1×10¹⁰ to about 1×10¹⁴ viral genomes/mL (vg/mL).

52. The method of any one of claims 1-51, wherein greater than about 70% of the rAAV particles in the composition are full rAAV capsids.

53. The method of any one of claims 1-52, wherein greater than about 70% to about 95% of the rAAV particles in the composition are full rAAV capsids.

54. The method of any one of claims 1-53, wherein the rAAV particles in the composition have been purified using one or more purification steps.

55. The method of any one of claims 1-54, wherein the recombinant viral particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAV13 capsid, an AAV14 capsid, an AAV15 capsid, an AAV16 capsid, an AAVrh20 capsid, an AAV.rh39 capsid, an AAV.Rh74 capsid, an AAV.RHM4-1 capsid, an AAV.hu37 capsid, an AAV.Anc80 capsid, an AAV.Anc80L65 capsid, an AAV.PHP.B capsid, an AAV2.5 capsid, an AAV2tYF capsid, an AAV3B capsid, an AAV.LK03 capsid, an AAV.HSC1 capsid, an AAV.HSC2 capsid, an AAV.HSC3 capsid, an AAV.HSC4 capsid, an AAV.HSC5 capsid, an AAV.HSC6 capsid, an AAV.HSC7 capsid, an AAV.HSC8 capsid, an AAV.HSC9 capsid, an AAV.HSC1O capsid, an AAV.HSC11 capsid, an AAV.HSC12 capsid, an AAV.HSC13 capsid, an AAV.HSC14 capsid, an AAV.HSC15 capsid, an AAV.HSC16 capsid, an AAV2R471A capsid, an AAV2/2-7m8 capsid, an AAV DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid rAAV2/HBoV1 (chimeric AAV/human bocavirus virus 1).

56. The method of any one of claims 1-55, wherein the recombinant viral particle comprises an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, an AAV12 ITR, an AAV-13 ITR, an AAV-14 ITR, an AAV-15 ITR, an AAV-16 ITR, an AAV.rh20 ITR, an AAV.rh39 ITR, an AAV.rh74 ITR, an AAV.rhM4-1 ITR, an AAV.hu37 ITR, an AAV.Anc80 ITR, an AAV DJ ITR, a goat AAV ITR, a bovine AAV ITR, or a mouse AAV ITR.

57. The method of any one of claims 1-56, wherein the rAAV genome is 2500 bases to 5500 bases in length.

58. The method of any one of claims 1-57, wherein the recombinant viral particles comprise a self-complementary AAV (scAAV) genome.

59. The method of any one of claims 6-58, wherein the rAAV particles are selected from the group consisting of rAAV5 particles and rAAV1 particles.

60. The method of claim 59, wherein the rAAV particles are rAAV5 particles.

61. The method of claim 60, wherein the rAAV particles are rAAV5 and the relative amount of empty capsids is calculated from the equation PA260/PA230=0.0057 (relative percent full capsids)+0.1137.

62. The method of claim 59, wherein the rAAV particles are rAAV1 particles.

63. The method of claim 62, wherein the rAAV particles are rAAV1 and the relative amount of empty capsids is calculated from the equation PA260/PA230=0.0054 (relative percent full capsids)+0.0886.

64. A method of monitoring the removal of empty capsids during the purification of a composition of rAAV particles, the method comprising collecting a sample of the composition before and following one or more steps of the purification process and analyzing each collected sample for the relative amount of empty capsids according to the method of any one of claims 1-63, wherein a decrease in the relative amount of empty capsids between the samples subsequently collected indicates removal of empty capsids from the preparation of rAAV particles.

65. A method of monitoring the removal of empty capsids during the purification of a composition of rAAV particles, the method comprising:
a) determining the relative amount of empty capsids according to the method of any one of claims 1-63 in a first sample collected before the purification process or before a step of the purification of the process,
b) determining the relative amount of empty capsids according to the method of any one of claims 1-37 in a second sample collected following the purification process or a step of the purification of the process,
wherein a decrease in the relative amount of empty capsids between the second and first collected samples indicates removal of empty capsids from the preparation of rAAV particles.

66. A kit for measuring the relative amount empty capsids in a composition of rAAV particles according the methods of any one of claims 1-65.

67. The kit of claim 66, wherein the kit comprises chromatography columns and/or buffers for use in the methods of any one of claims 1-65.

68. The kit of claim 66 or 67, wherein the kit comprises three or more reference standards wherein the three or more reference standards comprise known ratios of full capsids to empty capsids ranging from 1:0 to 0:1.
